(19) United States
(12) Patent Application Publication
(10) Pub. No.: US 2026/0027117 A1
(43) Pub. Date: Jan. 29, 2026

(54) METHODS OF TREATING SMALL CELL LUNG CANCER

(71) Applicant: Debiopharm International S.A., Lausanne (CH)

(72) Inventors: Esteban Rodrigo Imedio, Madrid (ES); Luke Piggott, Pully (CH); Anne Bellon, Pully (CH); Valerie Nicolas, St-Prex (CH); Anne Vaslin Chessex, Prilly (CH); Lars Damstrup, Hilleroed (DK); Claudio Zanna, Bedoin (FR)

(73) Assignee: Debiopharm International S.A., Lausanne (CH)

(21) Appl. No.: 18/844,741

(22) PCT Filed: Mar. 7, 2023
(86) PCT No.: PCT/EP2023/055751

§ 371 Date: Sep. 6, 2024

(30) Foreign Application Priority Data
Mar. 7, 2022 (EP) 22160556.1

(51) Int. Cl. A61K 31/519; A61K 31/555; A61K 31/7048; A61P 35/00

(52) U.S. Cl. CPC A61K 31/519; A61K 31/555; A61K 31/7048; A61P 35/00

(57) ABSTRACT

Methods of treating small cell lung cancer (SCLC) using a WEE1 inhibitor are provided.

### FIELD OF THE INVENTION

The present invention generally relates to the treatment of small cell lung cancer (SCLC). More specifically, the present invention relates to the use of a WEE1 inhibitor to treat SCLC in patients in need thereof.

### BACKGROUND

Lung cancer is the most common cancer worldwide with approximately 2.2 million new diagnoses and 1.8 million deaths in 2020, which corresponds to the second highest incidence among cancers and the most common cancer-related mortality. The World Health Organization (WHO) divides lung cancer into 2 major classes based on its biology, therapy, and prognosis: small cell lung cancer (SCLC) and non-small cell lung cancer (NSCLC).

SCLC accounts for approximately 15% of all newly diagnosed lung cancers. SCLC is an aggressive high-grade neuroendocrine tumor associated with a short doubling time, a high growth fraction, and early development of widespread metastases, which contribute to the extremely poor survival outcomes. At the time of diagnosis, approximately one third of patients with SCLC have a tumor confined to the ipsilateral chest, mediastinum, and supraclavicular nodes, designated limited disease (LD-SCLC), while two thirds of patients have a more widespread, extensive disease (ED-SCLC), leading to an even worse prognosis. The median survival of patients diagnosed with LD-SCLC is less than 2 years, while that of patients diagnosed with ED-SCLC is less than one year.

Combination chemotherapy is currently considered standard first-line therapy for ED-SCLC. Surgery and/or chemoradiotherapy are standard options for LD-SCLC. Platinum-based chemotherapies, including platinum drugs such as cisplatin or carboplatin are the most common chemotherapy regimens, in combination with other agents such etoposide or irinotecan, for a duration of 4 to 6 months. More recently, immunotherapy (e.g. atezolizumab, durvalumab) is sometimes being incorporated as part of the first-line therapy for some patients, in combination with standard chemotherapy, and it is likely to become one of the potential standards of care. Unfortunately, despite initially high response rates to first-line chemotherapy, long-term survival is unusual because patients commonly progress or relapse to first-line therapies (about 90% relapse rate).

In second-line therapy, i.e. for SCLC patients who relapse or progress after first-line therapy, the available therapeutic options vary depending on the timing to failure from standard first-line platinum-based chemotherapy. For patients in which SCLC relapses, recurs or progresses while on treatment, or within 90 days after the last dose of platinum therapy, therapeutic options in second-line are limited. These patients may receive topotecan, lurbinectedin (as of February 2022, available in the US only under FDA accelerated approval), sometimes other chemotherapies such as CAV (cyclophosphamide, doxorubicin—also known as Adriamycin—, vincristine) treatment, and potentially immunotherapies. Patients in which SCLC relapses, recurs or progresses more than 90 days after the last dose of platinum therapy, may benefit from the above-mentioned therapeutic options, in addition to a re-challenge with platinum-based chemotherapy treatment. Even with second-line therapy, the survival of patients with relapsed SCLC remains limited.

SCLC may also present a high mutational burden and genomic instability. The WEE1 tyrosine kinase is activated upon DNA damage and regulates the G2-M and S phase cell cycle checkpoints. Inhibition of WEE1 in the DNA repair pathway, such as in conjunction with genetic alterations and/or addition of a DNA damaging agent, results in mitotic catastrophe and apoptosis of cancer cells, offering an attractive approach to treating cancer. However, preliminary data in different patient populations of SCLC have shown limited antitumor activity of the WEE1 inhibitor adavosertib, for example in combination with olaparib (Li B et al, Abstract 1785P, ESMO 2020—Annals of Oncology (2020) 31 (suppl_4): S974-S987. 10.1016/annonc/annonc290), or in combination with carboplatin in platinum-refractory SCLC (BALTIC study, Arm B, NCT02937818).

These aspects of the SCLC disease and patient populations, as well as the limited success of current standard treatments available, highlight the unmet medical need for the development of new therapies for SCLC.

### SUMMARY OF THE INVENTION

The present invention relates to a WEE1 inhibitor for use in, or for use in the preparation of a medicament for, treating small cell lung cancer (SCLC) in a patient in need thereof, as well as to methods of treating SCLC in a patient in need thereof, comprising administering a therapeutically effective amount of a WEE1 inhibitor.

In some aspects of the uses or methods according to the present invention, the WEE1 inhibitor is a compound of formula (I)

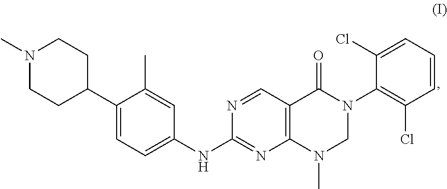 [SMILES: Cc1cc(Nc2ncc3c(n2)N(C)CN(c2c(Cl)cccc2Cl)C3=O)ccc1C1CCN(C)CC1]

or a pharmaceutically acceptable salt thereof.

In other aspects, the WEE1 inhibitor is any compound described in the patent applications WO2018090939, WO2022155202, WO2022256680, WO2013126656 and WO2008153207, each of which is fully incorporated herein by reference. Specifically, the WEE1 inhibitor may be a compound of one of the following formulas, or a pharmaceutically acceptable salt thereof:

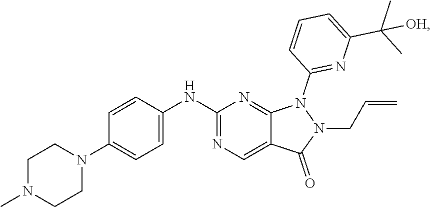 [SMILES: C=CCn1c(=O)c2cnc(Nc3ccc(N4CCN(C)CC4)cc3)nc2n1-c1cccc(C(C)(C)O)n1]

also known as AZD-1775 (adavosertib);

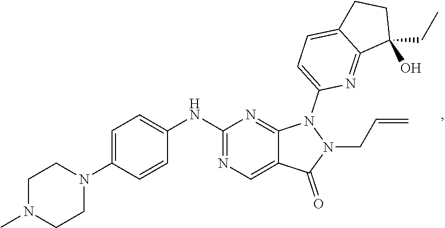 [SMILES: C=CCn1c(=O)c2cnc(Nc3ccc(N4CCN(C)CC4)cc3)nc2n1-c1ccc2c(n1)[C@@](O)(CC)CC2]

also known as Zn-C3 (azenosertib);

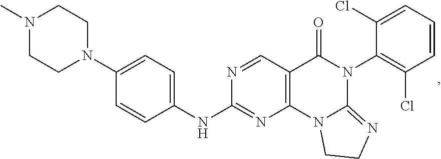 [SMILES: CN1CCN(c2ccc(Nc3ncc4c(n3)N3CCN=C3N(c3c(Cl)cccc3Cl)C4=O)cc2)CC1]

also known as 1MP7068 (as described in WO2018090939);

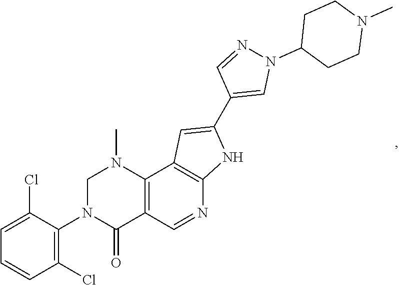 [SMILES: CN1CCC(n2cc(-c3cc4c5c(cnc4[nH]3)C(=O)N(c3c(Cl)cccc3Cl)CN5C)cn2)CC1]

also known as STC-81 23 (as described in WO20221 55202),

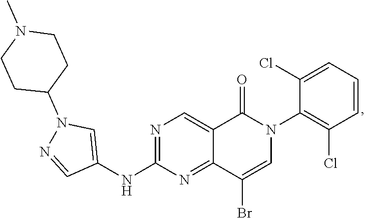 [SMILES: CN1CCC(n2cc(Nc3ncc4c(=O)n(-c5c(Cl)cccc5Cl)cc(Br)c4n3)cn2)CC1]

also known as ATRN-W1051 (as described in WO2022256680):

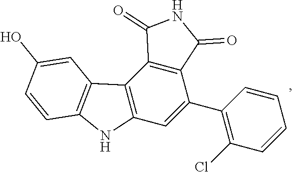 [SMILES: O=C1NC(=O)c2c1c(-c1ccccc1Cl)cc1[nH]c3ccc(O)cc3c21]

also known as PD0407824;

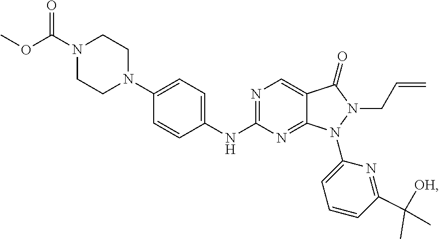 [SMILES: C=CCn1c(=O)c2cnc(Nc3ccc(N4CCN(C(=O)OC)CC4)cc3)nc2n1-c1cccc(C(C)(C)O)n1]

also known as CJM061;

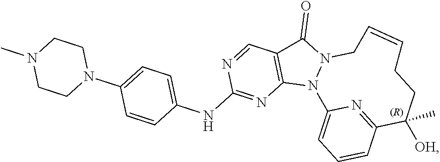

also known as SC0191;

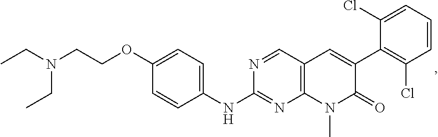 [SMILES: CCN(CC)CCOc1ccc(Nc2ncc3cc(-c4c(Cl)cccc4Cl)c(=O)n(C)c3n2)cc1]

also known as PD0166285;

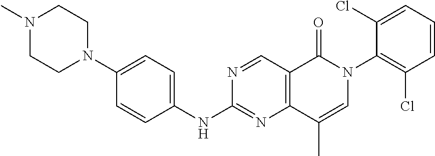 [SMILES: Cc1cn(-c2c(Cl)cccc2Cl)c(=O)c2cnc(Nc3ccc(N4CCN(C)CC4)cc3)nc12]

(as described in WO201 3126656), or

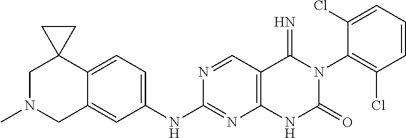 [SMILES: CN1Cc2cc(Nc3ncc4c(=N)n(-c5c(Cl)cccc5Cl)c(=O)[nH]c4n3)ccc2C2(CC2)C1]

(as described in WO2008153207).

In particularly preferred aspects of the uses and methods according to the present invention, the WEE1 inhibitor is a compound of formula (I), or a pharmaceutically acceptable salt thereof.

In the same or other aspects of the uses or methods according to the present invention, the WEE1 inhibitor is used in combination with carboplatin and/or etoposide. The WEE1 inhibitor may also be used in combination with carboplatin and etoposide.

In some aspects of the uses or methods according to the present invention, the SCLC has recurred or progressed after initial or prior SCLC treatment. For example, the SCLC has recurred or progressed 45 days or more after the last dose of standard platinum-based therapy, or the SCLC has recurred or progressed 90 days or more after the last dose of standard platinum-based therapy.

In some aspects of the uses or methods according to the present invention, the patient is naïve of any prior SCLC treatment.

In some aspects of the uses or methods according to the present invention, the WEE1 inhibitor is administered orally.

In some aspects of the uses or methods according to the present invention, the WEE1 inhibitor is administered on days 1, 2 and 3 of a 21-day cycle. The WEE1 inhibitor may also be administered on days 1, 2, 3 and 8, 9, 10 of a 21-day cycle.

In some aspects of the uses or methods according to the present invention, the WEE1 inhibitor is administered at a dose ranging from 150 to 720 mg per treatment day. For example, the WEE1 inhibitor may be administered as a single dose on a treatment day.

In some aspects of the uses or methods according to the present invention, the WEE1 inhibitor is administered at approximately the same time on each treatment day such as at the same time ±about 60 min, preferably ±60 min on each treatment day.

In some aspects of the uses or methods according to the present invention, the WEE1 inhibitor is administered after fasting, preferably for 4 hours. In the same or other aspects, WEE1 inhibitor administration may be followed by fasting, preferably for 2 hours.

In some aspects of the uses or methods according to the present invention, etoposide is administered by infusion. For example, etoposide is administered on days 1, 2 and 3 of a 21-day cycle. When administered by infusion, etoposide may be administered at a dose ranging from 70 to 100 mg/m2, preferably 100 mg/m2, per infusion.

In some aspects of the uses or methods according to the present invention, etoposide is administered orally. For example, etoposide is administered on days 1 to 5 of a 21-day cycle, at a dose ranging from 100 to 200 mg/m2/day. When administered orally, etoposide may also be administered at a dose ranging from 100 to 200 mg/m2/day on days 1 to 5 every 3 to 4 weeks, or 200 mg/m2/day on days 1, 3 and 5 every 3 to 4 weeks.

In some aspects of the uses or methods according to the present invention, carboplatin is administered by infusion, for example at a dose corresponding to an AUC ranging from 2 to 6 mg/ml×min, preferably 5 mg/ml×min, according to the Calvert formula. Carboplatin may be administered on day 1 of a 21-day cycle.

In some aspects of the uses or methods according to the present invention, on a WEE1 inhibitor treatment day, administration of the WEE1 inhibitor precedes administration of etoposide and/or carboplatin. For example, on a WEE1 inhibitor treatment day, etoposide administration is initiated about 5 minutes to 1 hour after WEE1 inhibitor administration. For example, on a WEE1 inhibitor treatment day, carboplatin administration is initiated about 5 minutes to 1 hour after WEE1 inhibitor administration, or after the end of etoposide administration.

In some aspects of the uses or methods according to the present invention, the WEE1 inhibitor is administered over 1, 2, 3, 4, 5, 6 or more cycles.

The present invention also relates to a pharmaceutical composition comprising a WEE1 inhibitor, in particular the compound of formula (I), for use in, or for use in the preparation of a medicament for, treating small cell lung cancer (SCLC) in a patient in need thereof.

The present invention also relates to a kit comprising a WEE1 inhibitor, in particular the compound of formula (I), for use in, or for use in the preparation of a medicament for, treating small cell lung cancer (SCLC) in a patient in need thereof. In some embodiments, the kit comprises a WEE1 inhibitor, in particular the compound of formula (I), as well as instructions for use in treating SCLC, in particular in accordance with the uses and methods described herein. Optionally, in specific embodiments, the kit may comprise, in separate containers, the WEE1 inhibitor, in particular the compound of formula (I), as well as etoposide or a pharmaceutically acceptable salt thereof and/or carboplatin.

The present invention also relates to etoposide or a pharmaceutically acceptable salt thereof for use in, or for use in the preparation of a medicament for, treating small cell lung cancer (SCLC) in a patient in need thereof, wherein the etoposide is used in combination with the compound of formula (I) or a pharmaceutically acceptable salt thereof, and optionally carboplatin, preferably wherein the use is as described further herein.

The present invention also relates to carboplatin or a pharmaceutically acceptable salt thereof for use in, or for use in the preparation of a medicament for, treating small cell lung cancer (SCLC) in a patient in need thereof, wherein the carboplatin is used in combination with the compound of formula (I) or a pharmaceutically acceptable salt thereof, and optionally etoposide or a pharmaceutically acceptable salt thereof, preferably wherein the use is as described further herein.

### BRIEF DESCRIPTION OF THE DRAWINGS

FIG. 1 is a diagram showing the Bliss synergy scores across a range of doses between compound of formula (I) (0-10 uM) and carboplatin (0-300 uM) in the in vitro assay described in Example 1.

FIG. 2 is a diagram showing mean tumour volumes as a function of time for each treatment group in the CDX in vivo model described in Example 2 where compound of formula (I) is administered alone or in combination with carboplatin.

FIG. 3 is a diagram showing mean tumour volumes as a function of time for each treatment group in the CDX in vivo model described in Example 2 where compound of formula (I) is administered alone or in combination with etoposide.

FIG. 4 is a diagram showing mean tumour volumes as a function of time for each treatment group in the CDX in vivo model described in Example 2 where compound of formula (I) is administered alone, in combination with etoposide, or in combination with both carboplatin and etoposide.

FIG. 5 is a diagram showing mean tumour volumes as a function of time for each treatment group in the CDX in vivo model described in Example 3 where compound of formula (I) is administered alone or in combination with carboplatin.

FIG. 6 is a diagram showing mean tumour volumes as a function of time for each treatment group in the PDX in vivo model described in Example 4 where compound of formula (I) is administered alone or in combination with etoposide.

FIG. 7 is a study diagram illustrating the schedule of administration of triple combination of compound of formula (I), carboplatin and etoposide in both the dose escalation (DE) and the expansion parts of the clinical trial described in Example 5b.

FIG. 8 is a diagram showing the Bliss synergy scores across a range of doses between compound of formula (I) and carboplatin/etoposide in 5 SCLC organoid models as described in Example 6.

### DETAILED DESCRIPTION OF THE INVENTION

The present invention relates to the use of a WEE1 inhibitor in the treatment of SCLC in patients in need thereof.

In some embodiments, the present invention relates to the use of a compound of formula (I)

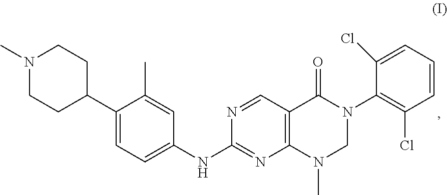 [SMILES: Cc1cc(Nc2ncc3c(n2)N(C)CN(c2c(Cl)cccc2Cl)C3=O)ccc1C1CCN(C)CC1]

or a pharmaceutically acceptable salt thereof, in the treatment of SCLC in patients in need thereof.

The present invention also relates to a method for treatment of SCLC in a patient in need thereof using a compound of formula (I) or a pharmaceutically acceptable salt thereof, as well as a method involving the combination treatments described herein. Any disclosure of a use of a WEE1 inhibitor in the treatment of SCLC may be understood as relating to the method for treatment of SCLC, and vice versa.

It has been found that compound of formula (I) synergized with carboplatin and with etoposide in vitro leading to a significant increase in the induction of cell death. In vivo, compound of formula (I) is efficacious in significantly improving SCLC tumor growth inhibition, in monotherapy, in combination with either carboplatin or etoposide, or in combination with both carboplatin and etoposide. For example, compound of formula (I) significantly improved the tumor growth inhibitory effect of etoposide and of carboplatin in both PDX and CDX models of SCLC in vivo. Also, triple combination of carboplatin, etoposide and compound of formula (I) resulted in significantly improved tumor response when compared to carboplatin and etoposide treatments alone or to the double combination of compound of formula (I) with either carboplatin or etoposide. Compound of formula (I) was also well tolerated either in monotherapy or in combination.

### Definitions

So that the invention may be more readily understood, certain terms are specifically defined below. Unless explicitly defined elsewhere in this document, all other technical and scientific terms used herein have the meaning that would be commonly understood by one of ordinary skill in the relevant art.

As used herein, including in the appended claims, the singular forms of words such as “a”, “an”, and “the”, include their corresponding plural references unless the context clearly indicates otherwise.

It is understood that wherever embodiments are described herein with the language “comprising,” otherwise analogous embodiments described in terms of “consisting of” and/or “consisting essentially of” are also provided.

The term “and/or” as used herein in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, a “WEE1 inhibitor” refers to a compound that inhibits the activity of the WEE1 kinase, for example with an IC50 of <10 nM in an ADP-GLO kinase assay or an IC50 of <100 nM in an enzyme profiling assay.

As used herein when referring to compound of formula (I), the expression “pharmaceutically acceptable salt” refers to salts prepared from pharmaceutically acceptable non-toxic acids including inorganic or organic acids. For example, acceptable salts derived from acids such as quaternary salt, acetate, carbonate, carbamate, sulfonate, strong inorganic acids and the like. In general, pharmaceutically acceptable salts may be used for modifying the solubility or hydrolysis characteristics of a compound, or in sustained release formulations. It will be understood that, as used herein, references to the compound of formula (I) are meant to also include the pharmaceutically acceptable salts unless stated otherwise.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

As used herein, administration “in combination with” one or more further therapeutic agents includes simultaneous (concurrent) and consecutive administration in any order.

As used herein, terms such as “treating” or “treatment” or “to treat” or “alleviating” or “to alleviate” refer to therapeutic measures that cure, slow down, lessen symptoms of, and/or halt or reverse progression or severity of a diagnosed pathologic condition, disorder or disease. Thus, those in need of treatment include those already diagnosed with or suspected of having the disorder. In certain embodiments, a subject is successfully “treated” for cancer according to the methods of the present invention if the patient shows one or more of the following: a reduction in the number of or complete absence of cancer cells; a reduction in the tumor size or burden; inhibition of or an absence of cancer cell infiltration into peripheral organs; inhibition of or an absence of tumor metastasis; inhibition of or an absence of tumor growth; relief of one or more symptoms associated with the specific cancer; reduced morbidity and mortality; improvement in quality of life; reduction in tumorigenicity, tumorigenic frequency, or tumorigenic capacity, of a tumor; reduction in the number or frequency of cancer stem cells in a tumor; differentiation of tumorigenic cells to a non-tumorigenic state; as well as increased chances to have a complete response (CR), a partial response (PR), increased chances to have the disease under control (e.g. CR, PR, stable disease SD), to live longer without progression, and without disease, to live longer, decreased chances to have a progressive disease (PD) and to increase the time until progression. Collectively for groups or populations of patients, successful treatment may result in endpoints such as increased Overall Response Rate (ORR), Best Overall Response (BOR), Duration of Response (DOR), Disease Control Rate (DCR), progression-free survival (PFS), overall survival (OS), time to progression (TTP) or any combination thereof.

As used herein, the term “SCLC treatment” refers to any treatment or therapy approved by at least one health authority such as FDA or EMA, or under clinical trial investigation to treat SCLC. “Initial SCLC treatment” refers to the first SCLC treatment administered to a given patient diagnosed with SCLC. “Prior” or “previous SCLC treatment” refers to one or more SCLC treatment administered to a given patient diagnosed with SCLC before the methods according to the present invention.

As used herein, the term “standard platinum-based (chemo)therapy” refers to the current standard of care (e.g. first-line) therapy for SCLC, namely a platinum drug such as cisplatin or carboplatin, in combination with other commonly used drugs for these regimens, such as etoposide or irinotecan. Such “standard platinum-based therapy” may also be used as second-line therapy, for example if prior first-line therapy with another agent, such as immunotherapy, has been used.

As used herein when referring to SCLC or to a patient suffering therefrom, the terms “relapse”, “to relapse”, “recurrence”, “to recur” mean a worsening of the disease and/or of the signs and symptoms of the disease after a period of improvement, stabilization or disease absence.

As used herein when referring to SCLC or to a patient suffering therefrom, the terms “progression” or “to progress” means when the cancer becomes worse, either due to existing lesions that are growing and/or due to appearance of new lesions.

As used herein, the term “treatment day” or “WEE1 inhibitor treatment day” refers to a day on which the WEE1 inhibitor is administered according to the methods of the present invention.

As used herein, the term “therapeutically effective amount” refers to an amount of a drug effective to “treat” a disease or disorder in a subject or patient. In the case of cancer, the therapeutically effective amount of the drug can reduce the number of cancer cells; reduce the tumor size or burden; inhibit (i.e., slow to some extent and in a certain embodiment, stop) cancer cell infiltration into peripheral organs; inhibit (i.e., slow to some extent and in a certain embodiment, stop) tumor metastasis; inhibit, to some extent, tumor growth; relieve to some extent one or more of the symptoms associated with the cancer; and/or result in a favorable response such as increased Overall Response Rate (ORR), Best Overall Response (BOR), Duration of Response (DOR), Disease Control Rate (DCR), progression-free survival (PFS), overall survival (OS), complete response (CR) rate, partial response (PR) rate, or, in some cases, stable disease (SD) rate, a decrease in progressive disease (PD), an increased time to tumor progression (TTP) or any combination thereof. See the definition herein of “treating”.

In some embodiments, the term “therapeutically effective amount” of a given drug when used in monotherapy, such as a “WEE1 therapeutically effective amount”, a “carboplatin therapeutically effective amount”, or an “etoposide therapeutically effective amount”, refers to an amount of a drug effective to “treat” a disease or disorder in a subject or patient, whilst keeping an acceptable safety profile. In some aspects, when administering a WEE1 inhibitor and carboplatin in combination, their respective therapeutically effective amounts may be referred to as the “WEE1 carbo-combination therapeutically effective amount” and the “carboplatin combination therapeutically effective amount”. In some aspects, when administering a WEE1 inhibitor and etoposide in combination, their respective therapeutically effective amounts may be referred to as the “WEE1 etopo-combination therapeutically effective amount” and the “etoposide combination therapeutically effective amount”. In some aspects, when administering a WEE1 inhibitor, carboplatin and etoposide in combination, their respective therapeutically effective amounts may be referred to as the “WEE1 triple combination therapeutically effective amount”, “carboplatin triple combination therapeutically effective amount” and “etoposide triple combination therapeutically effective amount”. In cases where the WEE1 inhibitor is compound of formula (I), the “WEE1 therapeutically effective amount” of the compound of formula (I) when administered as monotherapy may be different from the “WEE1 carbo-combination therapeutically effective amount”, may be different from the “WEE1 etopo-combination therapeutically effective amount”, and each may also be different from the, “WEE1 triple combination therapeutically effective amount” of the compound of formula (I) when used in combination. Similar considerations apply to the therapeutic effective amounts of carboplatin and etoposide, depending on whether they are used in monotherapy, in combination with the compound of formula (I), or in the triple combination. Similar considerations also apply to other WEE1 inhibitors or combinations thereof. In the case of cancer, the therapeutically effective amount of the drug can reduce the number of cancer cells; reduce the tumor size or burden; inhibit (i.e., slow to some extent and in a certain embodiment, stop) cancer cell infiltration into peripheral organs; inhibit (i.e., slow to some extent and in a certain embodiment, stop) tumor metastasis; inhibit, to some extent, tumor growth; relieve to some extent one or more of the symptoms associated with the cancer; and/or result in a favorable response such as increased Overall Response Rate (ORR), Best Overall Response (BOR), Duration of Response (DOR), Disease Control Rate (DCR), progression-free survival (PFS), overall survival (OS), complete response (CR) rate, partial response (PR) rate, or, in some cases, stable disease (SD) rate, a decrease in progressive disease (PD), an increased time to tumor progression (TTP) or any combination thereof. See the definition herein of “treating”.

As used herein, “RECIST v1.1” or “RECIST 1.1 criteria” refers to the “New response evaluation criteria in solid tumours, Revised RECIST guideline (version 1.1)” set out in Eisenhauer E. A. et aL., European Journal of Cancer 45 (2009) 228-247. It is to be understood that such RECIST guideline may evolve in the future and (a) new version(s) released.

As used herein, “Progression free survival” (PFS) in a clinical trial refers to the time from enrollment, first administration or randomization until disease progression or death from any cause, whichever occurs first. PFS is generally measured using the RECIST 1.1 criteria, and generally summarized using the Kaplan-Meier method.

As used herein, “Time to Tumor Progression” (TTP) in a clinical trial refers to the time from enrollment, first administration or randomization to disease progression. TTP is generally measured using the RECIST 1.1 criteria.

As used herein, a “complete response” or “complete remission” or “CR” in a clinical trial indicates that there is no detectable evidence of tumor in response to treatment.

This does not always mean the cancer has been cured. Complete response in solid tumors such as SCLC is generally measured using the RECIST 1.1 criteria.

As used herein, a “partial response” or “PR” in a clinical trial refers to a decrease in the size or volume of one or more tumors or lesions, or in the extent of cancer in the body, in response to treatment according to the RECIST 1.1 criteria.

As used herein, a “Stable disease” or “SD” in a clinical trial refers to disease without progression or relapse. In stable disease there is neither sufficient tumor shrinkage to qualify for partial response nor sufficient tumor increase to qualify as progressive disease taking as reference the smallest sum diameters while on the study. Generally measured using the RECIST 1.1 criteria.

As used herein, “Progressive disease” or “PD” in a clinical trial or study refers to the appearance of one or more new lesions or tumors and/or the unequivocal progression of existing target and/or non-target lesions and/or at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression). Generally measured using the RECIST 1.1 criteria.

As used herein, “Objective Response Rate” refers to the percentage of subjects in a study, clinical trial or treatment group who have a partial or complete response to the treatment. Generally measured using the RECIST 1.1 criteria.

As used herein, “Overall Response” or “Objective Response” refers to the response to treatment of a given patient in a study, clinical trial or treatment group at a given assessment point. Generally measured using the RECIST 1.1 criteria.

As used herein, “Best Overall Response” refers to the best response recorded for a given patient in a study, clinical trial or treatment group from the baseline assessment (enrollment, start of the treatment or randomization) until disease progression/recurrence (taking as reference for progressive disease the smallest measurements recorded since the treatment started) or death from any cause. Generally measured using the RECIST 1.1 criteria.

As used herein, “Disease Control Rate” refers to the percentage of patients in a study, clinical trial or treatment group who have achieved complete response, partial response or stable disease to a therapeutic intervention. Generally measured using the RECIST 1.1 criteria.

As used herein, “Duration of response” (DoR) refers to the time from earlier response (PR or better) to disease progression or death from any cause. Generally measured using the RECIST 1.1 criteria.

As used herein, “Overall Survival” (OS) in a clinical trial refers to the time from patient enrollment, first treatment administration or randomization to death from any cause or censored at the date last known alive. Improvement in OS includes a prolongation in life expectancy as compared to naive or untreated individuals or patients. Overall survival refers to the situation wherein a patient remains alive for a defined period of time, such as one year, five years, etc., e.g., from the time of randomization or first treatment.

As used herein, the term “pharmaceutical formulation” or “pharmaceutical composition” refers to a preparation which is in such form as to permit the biological activity of the active ingredient to be effective, and which contains no additional components which are unacceptably toxic to a subject to which the formulation would be administered.

As used herein, the expression “oral dosage form” refers to any form of a pharmaceutical composition that is suitable for oral administration.

### Methods of Treatment

In an embodiment of the present invention, the WEE1 inhibitor is used for the treatment of SCLC, in monotherapy.

In another embodiment of the present invention, the WEE1 inhibitor is used for the treatment of SCLC, in combination with carboplatin.

In yet another embodiment of the present invention, the WEE1 inhibitor is used for the treatment of SCLC, in combination with etoposide.

In yet another embodiment of the present invention, the WEE1 inhibitor is used for the treatment of SCLC, in combination with carboplatin and etoposide.

In any one of the embodiments of the present invention as described herein, the WEE1 inhibitor may be compound of formula (I)

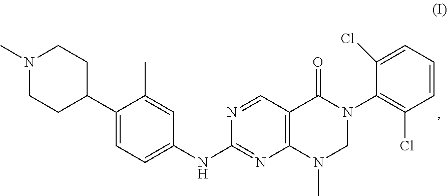 [SMILES: Cc1cc(Nc2ncc3c(n2)N(C)CN(c2c(Cl)cccc2Cl)C3=O)ccc1C1CCN(C)CC1]

or a pharmaceutically acceptable salt thereof.

In some embodiments of the methods of the present invention, the SCLC has recurred or progressed after initial SCLC treatment. In some embodiments of the methods of the present invention, the SCLC has recurred or progressed after prior SCLC treatment.

In more specific embodiments, the SCLC has recurred or progressed 45 days or more since the last dose of standard platinum-based therapy.

In other more specific embodiments, the SCLC has recurred or progressed 90 days or more since the last dose of standard platinum-based therapy.

In still other more specific embodiments, the SCLC has recurred or progressed 180 days or more since the last dose of standard platinum-based therapy.

In alternative embodiments, the patient is naïve of any previous SCLC treatment.

In some embodiments, the methods of treatment according to the present invention present advantageous properties in increasing anti-tumor activity (e.g. ORR, BOR, DOR and/or DCR) and/or time to event outputs (e.g. PFS and/or OS) in SCLC, for example as assessed per RECIST v1.1, with an acceptable safety profile and no detrimental effect on the patient's quality of life compared with e.g. re-challenge with platinum-based chemotherapy.

### Doses, Schedules and Routes of Administration

### WEE1 Inhibitor

In the methods of the present invention, the WEE1 inhibitor is used in a therapeutically effective amount for the intended purpose. Such amount may vary, for example depending on whether the WEE1 inhibitor is used in monotherapy or in combination with etoposide, with carboplatin or with both.

In some embodiments, a WEE1 therapeutically effective amount, a WEE1 carbo-combination therapeutically effective, a WEE1 etopo-combination therapeutically effective amount or a WEE1 triple combination therapeutically effective amount may be administered.

As used below, the term “about” describes a deviation from the indicated value of ±10%. The individual values may be understood as describing ranges between any of the described values.

The WEE1 inhibitor may generally be administered at doses ranging from about 10 to about 1000 mg of free base per treatment day, preferably about 100 to about 720 mg of free base per treatment day.

In an embodiment of the present invention where the WEE1 inhibitor is compound of formula (I), such compound of formula (I) may be administered at doses ranging from about 30 to about 1000 mg of free base, preferably ranging from about 90 to about 720 mg, or about 100 to about 720 mg of free base, per treatment day, even more preferably ranging from about 100 to about 520 mg of free base, per treatment day.

For example, compound of formula (I) may be administered at a dose of about 30, about 60, about 75, about 90, about 100, about 120, about 130, about 150, about 200, about 220, about 250, about 260, about 300, about 320, about 350, about 360, about 400, about 420, about 450, about 460, about 500, about 520, about 550, about 600, about 620, about 650, about 700, about 720, about 750, about 800, about 820, about 850, about 900, about 920, about 950 or about 1000 mg of free base per treatment day. Preferably, the compound of formula (I) may be administered at a dose of 30, 60, 75, 90, 100, 120, 130, 150, 200, 220, 250, 260, 300, 320, 350, 360, 400, 420, 450, 460, 500, 520, 550, 600, 620, 650, 700, 720, 750, 800, 820, 850, 900, 920, 950 or 1000 mg of free base per treatment day.

In a more specific embodiment, the compound of formula (I) may be administered at doses of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day. Preferably, the compound of formula (I) may be administered at a dose of 30, 60, 75, 90, 100, 120, 150, 200, 250, 260, 300, 350, 360, 400, 450, 460, 500, 520, 550 or 720 mg of free base per treatment day.

In another specific embodiment, the compound of formula (I) may be administered at doses of about 100, about 150, about 200, about 250, about 300, about 350, about 400, about 450, about 500, about 520, about 550, about 600, about 620, about 650, about 700, about 720, about 750, about 800, about 820, about 850, about 900, about 920, about 950 or about 1000 mg of free base per treatment day. In a more specific embodiment, compound of formula (I) may be administered at doses of about 100, about 150, about 200, about 300, about 400 or about 520 mg of free base per treatment day.

In a preferred embodiment of the present invention where the WEE1 inhibitor is compound of formula (I), such compound of formula (I) may be administered at doses ranging from 100 to 1000 mg of free base, preferably ranging from 150 to 720 mg of free base, per treatment day. For example, compound of formula (I) may be administered at a dose of 100, 150, 200, 250, 300, 350, 400, 450, 500, 520, 550, 600, 620, 650, 700, 720, 750, 800, 820, 850, 900, 920, 950 or 1000 mg of free base per treatment day. In a more specific embodiment, compound of formula (I) may be administered at doses of 100, 150, 200, 300, 400, 520 or 720 mg of free base per treatment day.

In some embodiments of the present invention, the WEE1 inhibitor is administered orally.

In some embodiments of the present invention, the WEE1 inhibitor is administered as a single dose per treatment day (QD), or in two doses per treatment day (BID).

In some embodiments, the WEE1 inhibitor is administered on Days 1, 2 and 3 of a 21-day cycle. In other embodiments of the present invention, the WEE1 inhibitor is administered on Days 1, 2, 3 and 8, 9, 10 of a 21-day cycle. In some embodiments, if the WEE1 inhibitor, especially the compound of formula (I), is administered on days 1, 2, 3 and 8, 9, 10 of a 21-day cycle, it is optionally possible to administer the WEE1 inhibitor, especially the compound of formula (I), only on days 1, 2 and 3 of subsequent 21-day cycles. This change of the administration days may be advantageous especially if the patient develops side-effects upon administration on days 1,2,3 and 8,9,10.

In some embodiments, the WEE1 inhibitor may be administered on Days 1, 2 and 3 of a 21-day cycle at the doses per treatment day indicated above. In some embodiments, the WEE1 inhibitor may be administered on Days 1, 2, 3 and 8, 9, 10 of a 21-day cycle at the doses per treatment day indicated above.

In these embodiments, the WEE1 inhibitor may be administered over 1, 2, 3, 4, 5, 6 or more cycles. Typically, there are no breaks between any consecutive cycles, i.e. the day following a prior 21-day cycle may be the first day of the consecutive cycle. In some aspects, the treatment with the WEE1 inhibitor such as compound of formula (I) may continue until progression of disease, unacceptable toxicity, patient's decision to stop, discontinuation as per physician's decision, initiation of subsequent antineoplastic treatment, the end of a clinical study, or death.

In some embodiments of the present invention, the WEE1 inhibitor is administered at approximately the same time on each treatment day, e.g. at the same time±about 60 min, preferably ±60 min on each treatment day, for example in a given cycle. In more specific embodiments, the WEE1 inhibitor is administered in the morning, for example between 5 a.m and noon.

In some embodiments of the present invention, the WEE1 inhibitor is administered after the patient has fasted, preferably for at least 4 hours. In the same or other embodiments, a fasting follows administration of the WEE1 inhibitor, preferably of at least 2 hours.

In alternative aspects of the uses and methods according to the present invention, the WEE1 inhibitor is administered in a fed state.

In yet other alternative aspects of the uses or methods according to the present invention, the WEE1 inhibitor is administered irrespective of food status of the patient.

### Etoposide

In some embodiments of the methods of the present invention, etoposide is used in a therapeutically effective amount for the intended purpose. Such amount may vary, for example depending on whether it is used in combination with the WEE1 inhibitor only or with the WEE1 inhibitor and carboplatin.

In some embodiments, an etoposide therapeutically effective amount, an etoposide combination therapeutically effective or an etoposide triple combination therapeutically effective amount may be administered.

In some embodiments, etoposide may be administered on the same days as the WEE1 inhibitor, such as on the same days of the 21-day cycle of the WEE1 inhibitor, or in a different schedule, as defined below.

In some embodiments of the present invention, etoposide is administered by intravenous (IV) infusion. In more specific embodiments, etoposide is administered on days 1, 2 and 3 of a 21-day cycle. When administered by IV infusion, etoposide may be administered at a dose ranging from 70 to 100 mg/m2, preferably 100 mg/m2, per infusion.

In alternative embodiments of the present invention, etoposide is administered orally. In more specific embodiments, etoposide may be administered, at a dose ranging from 100 to 200 mg/m2/per day on days 1 to 5 of a 21-day cycle. In alternative more specific embodiments, etoposide is administered at a dose of 100 to 200 mg/m2/day on days 1 to 5 every 3 to 4 weeks, or 200 mg/m2/day on days 1, 3 and 5 every 3 to 4 weeks.

In some embodiments, it may be desired to express the amount of etoposide to be administered in mg per treatment day. In such embodiments, the daily dose may be calculated based on the indicated mg/m²/day and the body surface area (BSA) of the patient. The BSA may be determined by the skilled person by methods known to him, such as by the Du Bois method (see e.g. Dubois D, Dubois E F. A formula to estimate the approximate surface area if height and weight be known. Arch Intern Med. 1916; 17:863-871) or the Mosteller formula (see e.g. Mosteller R D. Simplified calculation of body-surface area. N Engl J Med 1987; 317:1098).

Of course, in some embodiments, etoposide may be used in the form of a pharmaceutically acceptable salt, such as but not limited to etoposide phosphate. In such embodiments, etoposide indications of dosages, amounts or concentrations herein, which are indications for the free base, may be suitably adapted in view of the molecular weight of the respective salt.

### Carboplatin

In some embodiments of the methods of the present invention, carboplatin is used in a therapeutically effective amount for the intended purpose. Such amount may vary, for example depending on whether it is used in combination with the WEE1 inhibitor only or with the WEE1 inhibitor and etoposide.

In some embodiments, a carboplatin therapeutically effective amount, a carboplatin combination therapeutically effective or a carboplatin triple combination therapeutically effective amount may be administered.

In some embodiments of the present invention, carboplatin is administered by intravenous (IV) infusion. In more specific embodiments, carboplatin is administered on day 1 of a 21-day cycle.

In some embodiments, carboplatin is administered by IV infusion at a dose corresponding to an AUC (area under the curve) ranging from 2 to 6 mg/ml×min, preferably 5 mg/ml×min, according to the Calvert formula.

The Calvert formula is commonly used to determine the optimal carboplatin dosage and can thus be readily determined by the skilled person. Since the myelotoxicity and clinical efficacy of carboplatin are inversely correlated with the clearance of the drug, which is correlated to the glomerular filtration rate (GFR), dosing of this agent can be made more accurate by taking GFR into account compared to dosing based solely upon the patients' body surface area. The GFR, and thus the clearance of carboplatin, differ in each patient irrespective of the body area. Consequently, some patients undergo a higher systemic exposure, expressed as the area under the plasma concentration/time curve (AUC), than others when dosages of carboplatin are given on the basis of the body surface area. A high AUC correlates with increased toxicity, thus increasing the risks of the treatment, but in the case of a low AUC the therapeutical efficacy decreases. This indicates that an individual dosing strategy may be advantageous to obtain the optimal AUC. The Calvert formula can be used to calculate the carboplatin dose accurately in order to obtain a target AUC.

The Calvert formula shown below may be used to determine carboplatin dosage:

$$Dose ( mg ) = target AUC ( mg / ml \times min ) \times [ {GFR}^{*} ml / min + 25 ml / min ]$$

*GFR is the Glomerular Filtration Rate, which may be estimated by calculated creatinine clearance, e.g. using Cockcroft-Gault Equation. The Cockcroft-Gault equation for estimating creatinine clearance (CrCI) is as follows: CrCI (male)=([140−age]×weight in kg)/(serum creatinine×72).

The GFR may also be estimated using the CKD/EPI creatinine equation of 2021:

Expressed as a single equation:

$${eGFR}_{cr} = 142 \times {min ( {S}_{cr} / κ , 1 )}^{α} \times {max ( {S}_{cr} / κ , 1 )}^{- 1.2} \times {0.9938}^{Age} \times 1.012 [ if female ]$$

where:
S_{cr}=standardized serum creatinine in mg/dL
K=0.7 (females) or 0.9 (males)
α=−0.241 (female) or −0.302 (male)
min(S_{cr}/k, 1) is the minimum of S_{cr}/k or 1.0
max(S_{cr}/k, 1) is the maximum of S_{cr}/k or 1.0
Age (years)
The 1.012 factor only applies for female subjects

The CKD/EPI creatinine equation of 2021 is described, for instance, on the internet page of the National Kidney Foundation, and background references are cited therein for further information, including Levey A S, et al., Ann Intern Med. 2009; 150(9):604-612; Levey A S, Stevens L A., Am J Kidney Dis. 2010; 55(4):622-627; Matsushita K, et al., Am J Kidney Dis. 2010; 55(4):648-659; White S L, et al., Am J Kidney Dis. 2010; 55(4):660-670; and Becker B N, et al., Am J Kidney Dis. 2009; 55(1):8-10.

In some embodiments where carboplatin is administered at a dose corresponding to an AUC ranging from 2 to 3 mg/ml×min, it may be administered at a more frequent schedule, such as weekly in a 21-day cycle. For instance, carboplatin may be administered at a dose corresponding to an AUC ranging from 2 to 3 mg/ml×min on days 1, 8 and 15 of a 21-day cycle.

### Combination Treatments Involving the WEE1 Inhibitor

In some embodiments of the present invention where the WEE1 inhibitor is used in combination with etoposide and/or carboplatin, administration of the WEE1 inhibitor precedes administration of etoposide and/or carboplatin, on a WEE1 inhibitor treatment day.

In some embodiments, on a WEE1 inhibitor treatment day, etoposide administration is initiated about 5 minutes to 1 hour after WEE1 inhibitor administration, preferably about 5 to 30 minutes, even preferably about 5 to 15 minutes.

In some embodiments of the present invention, on a WEE1 inhibitor treatment day, carboplatin administration is initiated about 5 minutes to 1 hour after WEE1 inhibitor administration, or after the end of etoposide administration, preferably about 5 to 30 minutes.

In some combination embodiments, dexamethasone and/or granisetron may be administered as antiemetic prophylaxis prior to administration of any treatment agent. For example, dexamethasone and/or granisetron may be administered at Day 1, 2 and 3 of each cycle, prior to administration of the WEE1 inhibitor.

In some embodiments of the present invention, the combination of the WEE1 inhibitor with either carboplatin or etoposide, or with both, shows a synergistic effect in treating SCLC.

In any embodiments of the above-mentioned combinations, the WEE1 inhibitor may be compound of formula (I). Particularly, in some embodiments, the combination of compound of formula (I) with either carboplatin or etoposide shows a synergistic effect in treating SCLC, along with an acceptable safety profile. Also, in some embodiments, the triple combination of compound of formula (I) with carboplatin and etoposide shows a synergistic effect in treating SCLC, along with an acceptable safety profile.

As used below, the term “about” describes a deviation from the indicated value of ±10%. The individual values may be understood as describing ranges between any of the described values.

In all the following aspects of the uses or methods according to the present invention wherein the WEE1 inhibitor is compound of formula (I), the dose administered of compound of formula (I) ranges from about 30 to about 1000 mg of free base, preferably from about 90 to 720 mg, or about 100 to about 720 mg of free base, even more preferably from about 100 to about 520 mg of free base, per treatment day.

For example, compound of formula (I) may be administered at a dose of about 30, about 60, about 75, about 90, about 100, about 120, about 130, about 150, about 200, about 220, about 250, about 260, about 300, about 320, about 350, about 360, about 400, about 420, about 450, about 460, about 500, about 520, about 550, about 600, about 620, about 650, about 700, about 720, about 750, about 800, about 820, about 850, about 900, about 920, about 950 or about 1000 mg of free base per treatment day. Preferably, the compound of formula (I) may be administered at a dose of 30, 60, 75, 90, 100, 120, 130, 150, 200, 220, 250, 260, 300, 320, 350, 360, 400, 420, 450, 460, 500, 520, 550, 600, 620, 650, 700, 720, 750, 800, 820, 850, 900, 920, 950 or 1000 mg of free base per treatment day.

In all the following aspects of the uses or methods according to the present invention wherein the WEE1 inhibitor is compound of formula (I), the compound of formula (I) may preferably be administered at doses of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day. Preferably, the compound of formula (I) may be administered at a dose of 30, 60, 75, 90, 100, 120, 150, 200, 250, 260, 300, 350, 360, 400, 450, 460, 500, 520, 550 or 720 mg of free base per treatment day.

In all the following aspects of the uses or methods according to the present invention wherein the WEE1 inhibitor is compound of formula (I), the compound of formula (I) may even more preferably be administered at doses of about 100, about 150, about 200, about 250, about 300, about 350, about 400, about 450, about 500, about 520, about 550, about 600, about 620, about 650, about 700, about 720, about 750, about 800, about 820, about 850, about 900, about 920, about 950 or about 1000 mg of free base per treatment day. In a more specific embodiment, compound of formula (I) may be administered at doses of about 100, about 150, about 200, about 300, about 400, about 520 or about 720 mg of free base per treatment day. In an even more specific embodiment, compound of formula (I) may be administered at doses of about 100, about 150, about 200, about 300, about 400 or about 520 mg of free base per treatment day.

In all the following aspects of the uses or methods according to the present invention wherein the WEE1 inhibitor is compound of formula (I), the compound of formula (I) may particularly preferably be administered at doses ranging from 100 to 1000 mg of free base, preferably ranging from 150 to 720 mg of free base, per treatment day. For example, compound of formula (I) may be administered at a dose of 100, 150, 200, 250, 300, 350, 400, 450, 500, 520, 550, 600, 620, 650, 700, 720, 750, 800, 820, 850, 900, 920, 950 or 1000 mg of free base per treatment day. In a more specific embodiment, compound of formula (I) may be administered at doses of 100, 150, 200, 300, 400, 520 or 720 mg of free base per treatment day. In an even more specific embodiment, compound of formula (I) may be administered at doses of 100, 150, 200, 300, 400 or 520 mg of free base per treatment day.

In some aspects of the uses or methods according to the present invention, the combined treatment schedule or regimen with etoposide may be defined as in the following alternative or complementary items:
a) WEE1 inhibitor administered on Days 1, 2 and 3 of a 21-day cycle or Days 1, 2, 3 and 8, 9, 10 of a 21-day cycle, in combination with etoposide administered, preferably by intravenous (IV) infusion, on days 1, 2 and 3 of the 21-day cycle;
b) Compound of formula (I) administered on Days 1, 2 and 3 of a 21-day cycle or Days 1, 2, 3 and 8, 9, 10 of a 21-day cycle, in combination with etoposide administered, preferably by intravenous (IV) infusion, on days 1, 2 and 3 of the 21-day cycle;
c) WEE1 inhibitor administered on Days 1, 2 and 3 of a 21-day cycle or Days 1, 2, 3 and 8, 9, 10 of a 21-day cycle, in combination with etoposide administered, preferably by intravenous (IV) infusion, on days 1, 2 and 3 of the 21-day cycle, wherein
   the WEE1 inhibitor is administered at a dose of about 10 to 1000 mg, preferably about 100 to about 720 mg of free base per treatment day;
d) Compound of formula (I) administered on Days 1, 2 and 3 of a 21-day cycle or Days 1, 2, 3 and 8, 9, 10 of a 21-day cycle, in combination with etoposide administered, preferably by intravenous (IV) infusion, on days 1, 2 and 3 of the 21-day cycle, wherein
   the compound of formula (I) is administered at the doses defined herein, preferably at a dose of about 100 to about 720 mg of free base per treatment day;
e) Compound of formula (I) administered on Days 1, 2 and 3 of a 21-day cycle or Days 1, 2, 3 and 8, 9, 10 of a 21-day cycle, in combination with etoposide administered by intravenous (IV) infusion on days 1, 2 and 3 of the 21-day cycle, wherein
   the compound of formula (I) is administered at the doses defined herein, preferably at a dose of about 100 to about 720 mg of free base per treatment day, and
   etoposide is administered at a dose ranging from 70 to 100 mg/m², preferably 100 mg/m², per infusion;
f) Compound of formula (I) administered on Days 1, 2 and 3 of a 21-day cycle or Days 1, 2, 3 and 8, 9, 10 of a 21-day cycle, in combination with etoposide administered, preferably orally, on days 1 to 5 of the 21-day cycle;
g) Compound of formula (I) administered on Days 1, 2 and 3 of a 21-day cycle or Days 1, 2, 3 and 8, 9, 10 of a 21-day cycle, in combination with etoposide administered, preferably orally, on days 1 to 5 of the 21-day cycle, wherein
   the compound of formula (I) is administered at the doses defined herein, preferably at a dose of about 100 to about 720 mg of free base per treatment day;
h) Compound of formula (I) administered on Days 1, 2 and 3 of a 21-day cycle or Days 1, 2, 3 and 8, 9, 10 of a 21-day cycle, in combination with etoposide administered, preferably orally, on days 1 to 5 of the 21-day cycle, wherein
   the compound of formula (I) is administered at the doses defined herein, preferably at a dose of about 100 to about 720 mg of free base per treatment day, and
   etoposide is administered at a dose ranging from 100 to 200 mg/m²/per day;
i) Compound of formula (I) administered on Days 1, 2 and 3 of a 21-day cycle or Days 1, 2, 3 and 8, 9, 10 of a 21-day cycle, in combination with etoposide administered, preferably orally, at a dose of 100 to 200 mg/m²/day on days 1 to 5 every 3 to 4 weeks, or 200 mg/m²/day on days 1, 3 and 5 every 3 to 4 weeks, wherein
   the compound of formula (I) is administered at the doses defined herein, preferably at a dose of about 100 to about 720 mg of free base per treatment day.

In some aspects of the uses or methods according to the present invention, the combined treatment schedule or regimen with carboplatin may be defined as in the following alternative or complementary items:
j) WEE1 inhibitor administered on Days 1, 2 and 3 of a 21-day cycle or Days 1, 2, 3 and 8, 9, 10 of a 21-day cycle, in combination with carboplatin administered, preferably by intravenous (IV) infusion, on day 1 of the 21-day cycle;
k) Compound of formula (I) administered on Days 1, 2 and 3 of a 21-day cycle or Days 1, 2, 3 and 8, 9, 10 of a 21-day cycle, in combination with carboplatin administered, preferably by intravenous (IV) infusion, on day 1 of the 21-day cycle;
l) WEE1 inhibitor administered on Days 1, 2 and 3 of a 21-day cycle or Days 1, 2, 3 and 8, 9, 10 of a 21-day cycle, in combination with carboplatin administered by intravenous (IV) infusion on day 1 of the 21-day cycle, wherein
   the WEE1 inhibitor is administered at a dose of about 10 to 1000 mg, preferably about 100 to about 720 mg of free base per treatment day;
m) Compound of formula (I) administered on Days 1, 2 and 3 of a 21-day cycle or Days 1, 2, 3 and 8, 9, 10 of a 21-day cycle, in combination with carboplatin administered by intravenous (IV) infusion on day 1 of the 21-day cycle, wherein
   the compound of formula (I) is administered at the doses defined herein, preferably at a dose of about 100 to about 720 mg of free base per treatment day;
n) Compound of formula (I) administered on Days 1, 2 and 3 of a 21-day cycle or Days 1, 2, 3 and 8, 9, 10 of a 21-day cycle, in combination with carboplatin administered by intravenous (IV) infusion on day 1 of the 21-day cycle, wherein
   the compound of formula (I) is administered at the doses defined herein, preferably at a dose of about 100 to about 720 mg of free base per treatment day, and
   carboplatin is administered at a dose corresponding to an AUC (area under the curve) ranging from 2 to 6 mg/ml×min, preferably 5 mg/ml×min, according to the Calvert formula;
o) Compound of formula (I) administered on Days 1, 2 and 3 of a 21-day cycle or Days 1, 2, 3 and 8, 9, 10 of a 21-day cycle, in combination with carboplatin administered by intravenous (IV) infusion, wherein
   the compound of formula (I) is administered at the doses defined herein, preferably at a dose of about 100 to about 720 mg of free base per treatment day, and
carboplatin is administered at a dose corresponding to an AUC ranging from 2 to 3 mg/ml×min, according to the Calvert formula.

In some aspects of the uses or methods according to the present invention, the triple combined treatment schedule or regimen with etoposide, when administered by intravenous (IV) infusion, and carboplatin, may be defined as in the following alternative or complementary items:
p) WEE1 inhibitor administered on Days 1, 2 and 3 of a 21-day cycle or Days 1, 2, 3 and 8, 9, 10 of a 21-day cycle, in combination with etoposide administered by intravenous (IV) infusion on days 1, 2 and 3 of the 21-day cycle, and carboplatin administered, preferably by intravenous (IV) infusion, on day 1 of the 21-day cycle;
q) Compound of formula (I) administered on Days 1, 2 and 3 of a 21-day cycle or Days 1, 2, 3 and 8, 9, 10 of a 21-day cycle, in combination with etoposide administered by intravenous (IV) infusion on days 1, 2 and 3 of the 21-day cycle, and carboplatin administered, preferably by intravenous (IV) infusion, on day 1 of the 21-day cycle;
r) WEE1 inhibitor administered on Days 1, 2 and 3 of a 21-day cycle or Days 1, 2, 3 and 8, 9, 10 of a 21-day cycle, in combination with etoposide administered by intravenous (IV) infusion on days 1, 2 and 3 of the 21-day cycle, and carboplatin administered, preferably by intravenous (IV) infusion, on day 1 of the 21-day cycle, wherein
   the WEE1 inhibitor is administered at a dose of about 10 to 1000 mg, preferably about 100 to about 720 mg of free base per treatment day;
s) Compound of formula (I) administered on Days 1, 2 and 3 of a 21-day cycle or Days 1, 2, 3 and 8, 9, 10 of a 21-day cycle, in combination with etoposide administered by intravenous (IV) infusion on days 1, 2 and 3 of the 21-day cycle, and carboplatin administered, preferably by intravenous (IV) infusion, on day 1 of the 21-day cycle, wherein
   the compound of formula (I) is administered at the doses defined herein, preferably at a dose of about 100 to about 720 mg of free base per treatment day;
t) Compound of formula (I) administered on Days 1, 2 and 3 of a 21-day cycle or Days 1, 2, 3 and 8, 9, 10 of a 21-day cycle, in combination with etoposide administered by intravenous (IV) infusion on days 1, 2 and 3 of the 21-day cycle, and carboplatin administered by intravenous (IV) infusion on day 1 of the 21-day cycle, wherein
   the compound of formula (I) is administered at the doses defined herein, preferably at a dose of about 100 to about 720 mg of free base per treatment day, and
   etoposide is administered at a dose ranging from 70 to 100 mg/m², preferably 100 mg/m², per infusion;
u) Compound of formula (I) administered on Days 1, 2 and 3 of a 21-day cycle or Days 1, 2, 3 and 8, 9, 10 of a 21-day cycle, in combination with etoposide administered by intravenous (IV) infusion on days 1, 2 and 3 of the 21-day cycle, and carboplatin administered by intravenous (IV) infusion, on day 1 of the 21-day cycle, wherein
   the compound of formula (I) is administered at the doses defined herein, preferably at a dose of about 100 to about 720 mg of free base per treatment day, and
   carboplatin is administered at a dose corresponding to an AUC (area under the curve) ranging from 2 to 6 mg/ml×min, preferably 5 mg/ml×min, according to the Calvert formula;
v) Compound of formula (I) administered on Days 1, 2 and 3 of a 21-day cycle or Days 1, 2, 3 and 8, 9, 10 of a 21-day cycle, in combination with etoposide administered by intravenous (IV) infusion on days 1, 2 and 3 of the 21-day cycle, and carboplatin administered by intravenous (IV) infusion on day 1 of the 21-day cycle, wherein
   the compound of formula (I) is administered at the doses defined herein, preferably at a dose of about 100 to about 720 mg of free base per treatment day,
   etoposide is administered at a dose ranging from 70 to 100 mg/m², preferably 100 mg/m², per infusion, and
   carboplatin is administered at a dose corresponding to an AUC (area under the curve) ranging from 2 to 6 mg/ml×min, preferably 5 mg/ml×min, according to the Calvert formula;
w) Compound of formula (I) administered on Days 1, 2 and 3 of a 21-day cycle or Days 1, 2, 3 and 8, 9, 10 of a 21-day cycle, in combination with etoposide administered by intravenous (IV) infusion on days 1, 2 and 3 of the 21-day cycle, and carboplatin administered by intravenous (IV) infusion on day 1 of the 21-day cycle, wherein
   the compound of formula (I) is administered at the doses defined herein, preferably at a dose of about 100 to about 720 mg of free base per treatment day, and
   carboplatin is administered at a dose corresponding to an AUC ranging from 2 to 3 mg/ml×min, according to the Calvert formula;
x) Compound of formula (I) administered on Days 1, 2 and 3 of a 21-day cycle or Days 1, 2, 3 and 8, 9, 10 of a 21-day cycle, in combination with etoposide administered by intravenous (IV) infusion on days 1, 2 and 3 of the 21-day cycle, and carboplatin administered by intravenous (IV) infusion on day 1 of the 21-day cycle, wherein
   the compound of formula (I) is administered at the doses defined herein, preferably at a dose of about 100 to about 720 mg of free base per treatment day,
   etoposide is administered at a dose ranging from 70 to 100 mg/m², preferably 100 mg/m², per infusion, and
   carboplatin is administered at a dose corresponding to an AUC ranging from 2 to 3 mg/ml×min, according to the Calvert formula.

In some aspects of the uses or methods according to the present invention, the triple combined treatment schedule or regimen with etoposide, when administered orally, and carboplatin may be defined as in the following alternative or complementary items:
y) WEE1 inhibitor administered on Days 1, 2 and 3 of a 21-day cycle or Days 1, 2, 3 and 8, 9, 10 of a 21-day cycle, in combination with etoposide administered orally on days 1 to 5 of the 21-day cycle, and carboplatin administered, preferably by intravenous (IV) infusion, on day 1 of the 21-day cycle;
z) Compound of formula (I) administered on Days 1, 2 and 3 of a 21-day cycle or Days 1, 2, 3 and 8, 9, 10 of a 21-day cycle, in combination with etoposide administered orally on days 1 to 5 of the 21-day cycle, and carboplatin administered, preferably by intravenous (IV) infusion, on day 1 of the 21-day cycle;
aa) WEE1 inhibitor administered on Days 1, 2 and 3 of a 21-day cycle or Days 1, 2, 3 and 8, 9, 10 of a 21-day cycle, in combination with etoposide administered orally on days 1 to 5 of the 21-day cycle, and carboplatin administered, preferably by intravenous (IV) infusion, on day 1 of the 21-day cycle, wherein
   the WEE1 inhibitor is administered at a dose of about 10 to 1000 mg, preferably about 100 to about 720 mg of free base per treatment day;
bb) Compound of formula (I) administered on Days 1, 2 and 3 of a 21-day cycle or Days 1, 2, 3 and 8, 9, 10 of a 21-day cycle, in combination with etoposide administered orally on days 1 to 5 of the 21-day cycle, and carboplatin administered, preferably by intravenous (IV) infusion, on day 1 of the 21-day cycle, wherein
   the compound of formula (I) is administered at the doses defined herein, preferably at a dose of about 100 to about 720 mg of free base per treatment day, and
cc) Compound of formula (I) administered on Days 1, 2 and 3 of a 21-day cycle or Days 1, 2, 3 and 8, 9, 10 of a 21-day cycle, in combination with etoposide administered orally on days 1 to 5 of the 21-day cycle, and carboplatin administered by intravenous (IV) infusion, on day 1 of the 21-day cycle, wherein
   the compound of formula (I) is administered at the doses defined herein, preferably at a dose of about 100 to about 720 mg of free base per treatment day, and
   etoposide is administered at a dose ranging from 100 to 200 mg/m²/per day;
dd) Compound of formula (I) administered on Days 1, 2 and 3 of a 21-day cycle or Days 1, 2, 3 and 8, 9, 10 of a 21-day cycle, in combination with etoposide administered orally on days 1 to 5 of the 21-day cycle, and carboplatin administered by intravenous (IV) infusion on day 1 of the 21-day cycle, wherein
   the compound of formula (I) is administered at the doses defined herein, preferably at a dose of about 100 to about 720 mg of free base per treatment day, and
   carboplatin is administered at a dose corresponding to an AUC (area under the curve) ranging from 2 to 6 mg/ml×min, preferably 5 mg/ml×min, according to the Calvert formula;
ee) Compound of formula (I) administered on Days 1, 2 and 3 of a 21-day cycle or Days 1, 2, 3 and 8, 9, 10 of a 21-day cycle, in combination with etoposide administered orally, and carboplatin administered by intravenous (IV) infusion on day 1 of the 21-day cycle, wherein
   the compound of formula (I) is administered at the doses defined herein, preferably at a dose of about 100 to about 720 mg of free base per treatment day,
   etoposide is administered at a dose of 100 to 200 mg/m²/day on days 1 to 5 every 3 to 4 weeks, or 200 mg/m²/day on days 1, 3 and 5 every 3 to 4 weeks, and
   carboplatin is administered at a dose corresponding to an AUC (area under the curve) ranging from 2 to 6 mg/ml×min, preferably 5 mg/ml×min, according to the Calvert formula;
ff) Compound of formula (I) administered on Days 1, 2 and 3 of a 21-day cycle or Days 1, 2, 3 and 8, 9, 10 of a 21-day cycle, in combination with etoposide administered orally on days 1 to 5 of the 21-day cycle, and carboplatin administered by intravenous (IV) infusion on day 1 of the 21-day cycle, wherein
   the compound of formula (I) is administered at the doses defined herein, preferably at a dose of about 100 to about 720 mg of free base per treatment day, and
   carboplatin is administered at a dose corresponding to an AUC ranging from 2 to 3 mg/ml×min, according to the Calvert formula;
gg) Compound of formula (I) administered on Days 1, 2 and 3 of a 21-day cycle or Days 1, 2, 3 and 8, 9, 10 of a 21-day cycle, in combination with etoposide administered orally, and carboplatin administered by intravenous (IV) infusion on day 1 of the 21-day cycle, wherein
   the compound of formula (I) is administered at the doses defined herein, preferably at a dose of about 100 to about 720 mg of free base per treatment day,
   etoposide is administered at a dose of 100 to 200 mg/m²/day on days 1 to 5 every 3 to 4 weeks, or 200 mg/m²/day on days 1, 3 and 5 every 3 to 4 weeks; and
   carboplatin is administered at a dose corresponding to an AUC ranging from 2 to 3 mg/ml×min, according to the Calvert formula.

It is also possible to use another WEE1 inhibitor instead of the compound of formula (I). In some embodiments the combined treatment schedule or regimen is therefore in accordance with anyone of embodiments a) to gg), but wherein a WEE1 inhibitor (any WEE1 inhibitor and especially the specific WEE1 inhibitors specified above) is used instead of compound of formula (I), as exemplified in embodiments a), c), j), l), p), r), y) and aa).

In some embodiments, the combined treatment schedule or regimen is in accordance with anyone of embodiments a) to gg), wherein the SCLC has recurred or progressed after initial SCLC treatment (second line treatment).

In some embodiments, the combined treatment schedule or regimen is in accordance with anyone of embodiments a) to gg), wherein the SCLC has recurred or progressed after prior SCLC treatment.

In some embodiments, the combined treatment schedule or regimen is in accordance with anyone of embodiments a) to gg), wherein the SCLC has recurred or progressed 45 days or more since the last dose of standard platinum-based therapy.

In some embodiments, the combined treatment schedule or regimen is in accordance with anyone of embodiments a) to gg), wherein the SCLC has recurred or progressed 90 days or more since the last dose of standard platinum-based therapy.

In some embodiments, the combined treatment schedule or regimen is in accordance with anyone of embodiments a) to gg), wherein the SCLC has recurred or progressed 180 days or more since the last dose of standard platinum-based therapy.

In some embodiments, the combined treatment schedule or regimen is in accordance with anyone of embodiments a) to gg), wherein the patient is naïve of any previous SCLC treatment (first line treatment).

In some embodiments, the combined treatment schedule or regimen is in accordance with anyone of embodiments a) to gg) and presents advantageous properties in increasing anti-tumor activity (e.g. ORR, BOR, DOR and/or DCR such as assessed per RECIST v 1.1) and/or time to event outputs (e.g. PFS and/or OS such as assessed per RECIST v 1.1) in SCLC, with an acceptable safety profile and no detrimental effect on the patient's quality of life compared with e.g. re-challenge with platinum-based chemotherapy.

In some embodiments, the combined treatment schedule or regimen is in accordance with anyone of embodiments a) to gg), wherein the WEE1 inhibitor, e.g. compound of formula (I) is administered as a single dose per treatment day (QD), or in two doses per treatment day (BID).

In some embodiments, the combined treatment schedule or regimen is in accordance with anyone of embodiments a) to gg), wherein the WEE1 inhibitor, e.g. compound of formula (I) is administered on Days 1, 2 and 3 of a 21-day cycle.

In some embodiments, the combined treatment schedule or regimen is in accordance with anyone of embodiments a) to gg), wherein the WEE1 inhibitor, e.g. compound of formula (I) is administered on Days 1, 2, 3 and 8, 9, 10 of a 21-day cycle.

In some embodiments, the combined treatment schedule or regimen is in accordance with anyone of embodiments a) to gg), wherein the WEE1 inhibitor, e.g. compound of formula (I) is administered over 1, 2, 3, 4, 5, 6 or more 21-day cycles.

In some embodiments, the combined treatment schedule or regimen is in accordance with anyone of embodiments a) to gg), wherein the WEE1 inhibitor, e.g. compound of formula (I) is administered at approximately the same time on each treatment day such as at the same time ±about 60 min, preferably ±60 min, for example in a given cycle.

In some embodiments, the combined treatment schedule or regimen is in accordance with anyone of embodiments a) to gg), wherein the WEE1 inhibitor, e.g. compound of formula (I) is administered in the morning, for example between 5 a.m and noon.

In some embodiments, the combined treatment schedule or regimen is in accordance with anyone of embodiments a) to gg), wherein the WEE1 inhibitor, e.g. compound of formula (I) is administered after the patient has fasted, preferably for at least 4 hours, or is administered in a fed state. In the same or other embodiments, a fasting follows administration of the WEE1 inhibitor, preferably of at least 2 hours.

In some embodiments, the combined treatment schedule or regimen is in accordance with anyone of embodiments a) to gg), wherein, if etoposide and/or carboplatin is administered on a WEE1 inhibitor treatment day, administration of the WEE1 inhibitor precedes administration of etoposide and/or carboplatin, on said WEE1 inhibitor treatment day.

In some embodiments, the combined treatment schedule or regimen is in accordance with anyone of embodiments a) to gg), wherein on a WEE1 inhibitor treatment day, if etoposide is administered on said WEE1 inhibitor treatment day, etoposide administration is initiated about 5 minutes to 1 hour after WEE1 inhibitor administration, preferably about 5 to 30 minutes, even preferably about 5 to 15 minutes.

In some embodiments, the combined treatment schedule or regimen is in accordance with anyone of embodiments a) to gg), wherein on a WEE1 inhibitor treatment day, if carboplatin and/or etoposide is administered on said WEE1 inhibitor treatment day, carboplatin administration is initiated about 5 minutes to 1 hour after WEE1 inhibitor administration, or after the end of etoposide administration, preferably about 5 to 30 minutes.

In some embodiments, the combined treatment schedule or regimen is in accordance with anyone of embodiments a) to gg), wherein dexamethasone and/or granisetron may be administered as antiemetic prophylaxis prior to administration of any treatment agent. For example, dexamethasone and/or granisetron may be administered at Day 1, 2 and 3 of each cycle, prior to administration of the WEE1 inhibitor.

In some embodiments, the combined treatment schedule or regimen is in accordance with anyone of embodiments a) to gg) and the WEE1 inhibitor, e.g. compound of formula (I) is administered orally.

In some embodiments, the combined treatment schedule or regimen is in accordance with anyone of embodiments a) to gg) and the compound of formula (I) is administered orally by means of a capsule as described herein.

In some embodiments, the combined treatment schedule or regimen is in accordance with anyone of embodiments a) to gg) and the WEE1 inhibitor, e.g. compound of formula (I) is administered orally in a dosage of from 100 to 520 mg of free base, per treatment day.

In some embodiments, the combined treatment schedule or regimen is in accordance with anyone of embodiments a) to gg) and the WEE1 inhibitor, e.g. compound of formula (I) is administered orally on an empty stomach.

In some embodiments, the combined treatment schedule or regimen is in accordance with anyone of embodiments a) to e) or p) to x) and on a WEE1 inhibitor treatment day, if etoposide is administered on said WEE1 inhibitor treatment day, etoposide is administered by infusion starting 5-15 min after the administration of the WEE1 inhibitor, e.g. compound of formula (I), at a dose of 100 mg/m², and infusion duration is 30 min to 1 h.

In some embodiments, the triple combined treatment schedule or regimen is in accordance with anyone of embodiments p) to x) and the carboplatin is administered by infusion starting within 30 min after the end of an etoposide infusion at a dose based on the AUC 5 mg/mL·min, and the infusion duration of carboplatin is 30 min to 1 h.

In some embodiments, the combined treatment schedule or regimen is in accordance with anyone of embodiments a) to gg), wherein treatment duration is until either progression of disease, unacceptable toxicity, or participant withdrawal.

In some embodiments, the patients to be treated are characterized by one or more of the inclusion and/or exclusion criteria specified in Examples 5a and/or 5b.

### Pharmaceutical Compositions

Pharmaceutical compositions of the present invention that are suitable for oral administration (oral dosage forms) may be presented in solid or liquid form. Suitable solid oral dosage forms include capsules, tablets, powders or granules and the like, each containing a predetermined amount of the active ingredient. Suitable liquid oral dosage forms include solutions, emulsions or suspensions. Pharmaceutical compositions of the present invention may also be in the form of sustained release formulations.

Any inert ingredient that is commonly used as a carrier or diluent may be used as pharmaceutically acceptable excipient in the solid oral formulations of the present invention, such as for example, a gum, a starch, a sugar, a cellulosic material, an acrylate, or mixtures thereof. Preferred diluents include, for example, microcrystalline cellulose, anhydrous lactose. The compositions may further comprise a disintegrating agent (e.g., croscarmellose sodium, sodium starch glycolate) and a lubricant (e.g., magnesium stearate), and may additionally comprise one or more additives selected from a binder (e.g., hydroxypropylcellulose), a glidant (e.g., silicon dioxide), a buffer (e.g., citric acid), a surfactant (e.g., tween 80), a solubilizing agent (e.g., cyclodextrin), a plasticizer (e.g., triacetin), an emulsifier (e.g, sodium lauryl sulfate), a stabilizing agent (e.g., povidone, ascorbic acid), a viscosity increasing agent (e.g., hydroxypropyl methylcellulose), a sweetener (e.g., sucrose), a film forming agent (e.g., cellulose based systems, polymers), a colorant (e.g., iron oxide), a flavoring agent or any combination thereof.

The oral pharmaceutical compositions of the present invention may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

### WEE1 Inhibitor

In some embodiments of the present invention, the WEE1 inhibitor is formulated in a pharmaceutical composition being an oral dosage form. In more specific embodiments, the WEE1 inhibitor is in the form of a solid oral dosage form, such as tablets or capsules (which may comprise powder or minitablets). Capsules may be soft or hard capsules.

For example, the WEE1 inhibitor is formulated in capsules, such as hard gelatin capsules comprising minitablets, each minitablet containing 10 mg of active ingredient. In some embodiments, the WEE1 inhibitor is a compound of formula (I). The following capsule strengths may be available: 20, 30, 60, 100, 130, 150 and 200 mg of active ingredient (free base). Intermediate capsule strengths may readily be produced to cover alternative dose levels.

In some embodiments, the solid oral dosage form is packaged in a blister and/or a bottle. In some embodiments, one, two, three, four, five or six unit dosage forms are administered per intake of the WEE1 inhibitor. Preferably, one or two unit dosage form(s) is administered per intake of the WEE1 inhibitor.

For example, when formulated as capsules containing minitablets, the excipients in each minitablet may include microcrystalline cellulose, anhydrous lactose, hydroxypropylcellulose, sodium starch glycolate, anhydrous colloidal silica and magnesium stearate. Alternative excipients may be used in an oral dosage form.

### Etoposide

In some embodiments of the present invention, etoposide is formulated in a pharmaceutical composition being a concentrate solution for infusion. In more specific embodiments, etoposide is in the form of a 20 mg/ml concentrate solution for infusion, so that each 5 ml vial contains 100 mg of etoposide, each 10 ml vial contains 200 mg of etoposide, each 12.5 ml vial contains 250 mg of etoposide, each 20 ml vial contains 400 mg of etoposide, each 25 ml vial contains 500 mg of etoposide, and each 50 ml vial contains 1000 mg of etoposide.

For example, a 20 mg/ml etoposide concentrate solution for infusion comprises benzyl alcohol and anhydrous ethanol (excipients with known effect).

In a concentrate etoposide solution for infusion embodiment, the full list of excipients may include anhydrous citric acid, benzyl alcohol, polysorbate 80, macrogol 300, and anhydrous ethanol. Alternative excipients may be used in a solution being bioequivalent.

In alternative embodiments of the present invention, etoposide is formulated in a pharmaceutical composition being an oral dosage form. In more specific embodiments, the pharmaceutical composition containing etoposide is a soft capsule. For example, each capsule may contain 50 mg etoposide as well as sodium ethyl parahydroxybenzoate (E215) and sodium propyl parahydroxybenzoate (E217) (excipients with known effects). Alternative excipients may be used in an oral dosage form being bioequivalent.

In a soft etoposide capsule embodiment, the capsule may contain: Citric acid, anhydrous (E330), Macrogol 400 (E1521), Glycerol (85 percent) (E422) and Water, purified. The capsule shell may contain: Glycerol (85 percent) (E422), Gelatin (E441), Sodium ethyl parahydroxybenzoate (E215), Sodium propyl parahydroxybenzoate (E217), Titanium dioxide (E171) and Red iron oxide (E172). Alternative excipients may be used in an oral dosage form being bioequivalent.

In some embodiments of the present invention, an oral dosage form, such as a solid oral dosage form, may incorporate both compound of formula (I) and etoposide as active ingredients.

Of course, in some embodiments, etoposide may be used in the form of a pharmaceutically acceptable salt, such as but not limited to etoposide phosphate. In such embodiments, etoposide indications of dosages, amounts or concentrations herein, which are indications for the free base, may be suitably adapted in view of the molecular weight of the respective salt.

### Carboplatin

In some embodiments of the present invention, carboplatin is formulated as a solution for intravenous (IV) infusion. In more specific embodiments, such solution is a carboplatin 10 mg/ml solution for infusion.

For example, the solution for infusion may comprise carboplatin and water for injection.

In use, a carboplatin solution for infusion may be further diluted in glucose 5% and administered as an intravenous infusion. Alternatively, a carboplatin solution for infusion may be further diluted in sodium chloride 0.9% and administered as an intravenous infusion.

Embodiments of the present disclosure can be further defined and illustrated by reference to the following non-limiting examples. It will be apparent to those skilled in the art that many modifications or changes, e.g. to the materials and methods described, can be practiced without departing from the scope of the present disclosure.

### EXAMPLES

### Example 1: In Vitro Synergy of Compound of Formula (I) in Combination with Carboplatin in SCLC Cell Line NCI-H446

NCI-H446 tumor cells were plated at a density of 5000 cells/90 ul in 96 well plates. Cells were treated with Compound of formula (I) and/or carboplatin, either alone or in combination with 9 different doses (3-fold serial dilutions starting at 10 uM for Compound of formula (I) and 300 uM for carboplatin) for 72 hours. Cell viability was then assessed by CellTiter-Glo viability assay and synergy of drug combination at each dose calculated using Bliss synergy method (e.g. Foucquier et al., 2015, Pharma Res Per, 3(3), 2015, e00149, doi: 10.1002/prp2.149; original reference Bliss C. I., Annals of Applied Biology, Volume 26, Issue 3, August 1939, Pages 585-615) where a score of >5 demonstrates synergy of the combination.

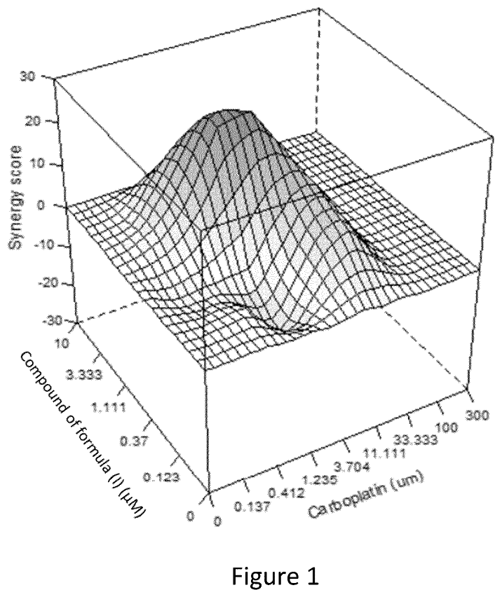

These in vitro studies demonstrate that Compound of formula (I) produces a strong synergistic antitumor activity (increased tumor cell death) in combination with carboplatin across a broad range of doses (FIG. 1).

Similar synergy was also observed in vitro with etoposide.

### Example 2: In Vivo Efficacy and Tolerability in NCI-H1048 Cell Line-Derived Xenoqraft (CDX) Model

Female Balb/c nude mice were inoculated subcutaneously with 5×10⁶ NCI-H1048 SCLC tumor cells. Animals were randomized when tumor volume reached approximately 100-150 mm³. Animals were treated with vehicle (0.5% methycellulose, 1% Tween 80 P.O. and saline I.V.), Compound of formula (I) formulated as a suspension in 0.5% methycellulose and 1% Tween 80 (30 mg/kg QD, P.O.), Carboplatin (50 mg/kg QW, I.V.) or etoposide (25 mg/kg or 12.5 mg/kg QW, I.P.), administered over a 21 day cycle, either alone or in combination (8 animals per group in double combination studies; 10 animals per group in triple combination study). In combination arms, Compound of formula (I) was administered 2 hours prior to chemotherapy. Tumor growth was monitored twice weekly by caliper measurement and tumor volume calculated using the equation V=(L×W×W)/2, where V is tumor volume, L is tumor length (the longest tumor dimension) and W is tumor width (the longest tumor dimension perpendicular to L). During routine monitoring, the animals were checked for any effects of tumor growth and treatments on behavior such as mobility, food and water consumption, body weight gain/loss 3 times a week, eye/hair matting and any other abnormalities. Mortality and observed clinical signs were recorded for individual animals.

No significant body weight loss was observed throughout the study, indicating that the administration of Compound of formula (I) in monotherapy, in combination with carboplatin, in combination with etoposide, or as a triple combination, was well tolerated.

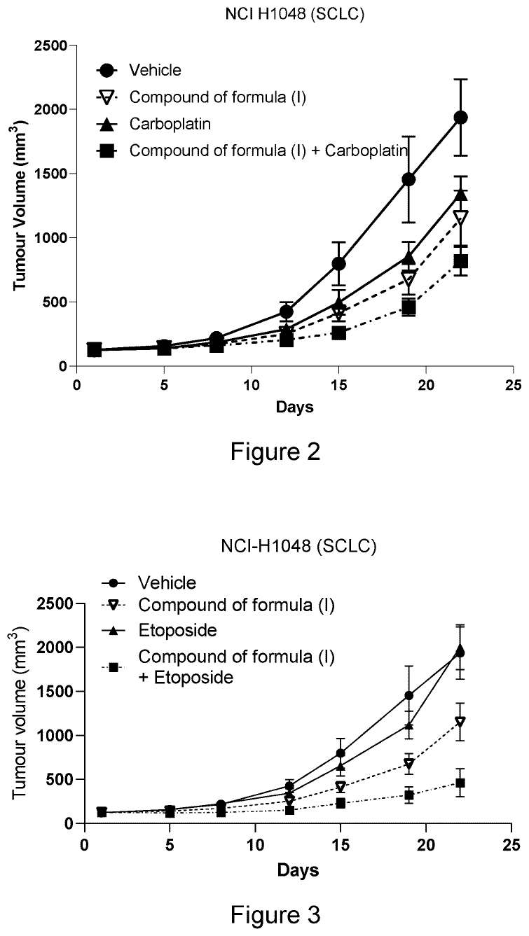

In repetitions of this NCI-H1048 tumor model, single agent (monotherapy) treatment with Compound of formula (I) led to a moderate but significant tumor growth inhibition (TGI) of up to 54% (p=0.016; see e.g. FIGS. 2 and 3). Combination treatment of Compound of formula (I) with either carboplatin (FIG. 2) or etoposide (FIG. 3) led to significant anti-tumor activity with TGI of about 68% (p=0.0026) and 78% (p=0.00015), respectively, compared to vehicle controls.

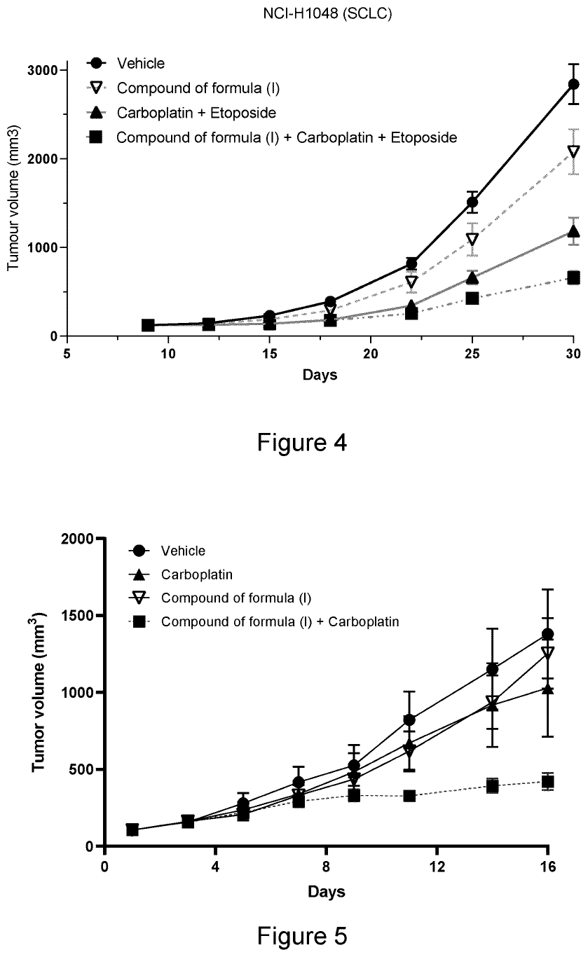

Furthermore, treatment of NCI-H1048 tumors with Compound of formula (I), carboplatin (50 mg/kg) and etoposide (12.5 mg/kg) also led to a significant anti-tumor activity resulting in a TGI of 72% compared to vehicle controls (p<0.0001) and a TGI of 44% when compared to tumors treated with the carboplatin and etoposide combination, thus demonstrating significant improvement in anti-tumor activity of the triple combination (Compound of formula (I), carboplatin and etoposide) compared to standard of care carboplatin+etoposide treatment (p=0.0043) (FIG. 4).

Thus, triple combination of carboplatin, etoposide and compound of formula (I) was well tolerated and resulted in significantly improved tumor response when compared to carboplatin and etoposide treatments alone or to the double combination.

### Example 3: In Vivo Efficacy and Tolerability in NCI-H446 CDX Model

Female Balb/c nude mice were inoculated subcutaneously with 5×10⁶ NCI-H446 SCLC tumor cells. Animals were randomized when tumor volume reached approximately 100-150 mm³. Animals were treated with vehicle (0.5% methycellulose, 1% Tween 80 P.O. and saline I.V.), Compound of formula (I) formulated as a suspension in 0.5% methycellulose and 1% Tween 80 (30 mg/kg QD, 3 consecutive days a week, P.O.), or Carboplatin (50 mg/kg QW, I.V.) administered over a 16 day cycle, either alone or in combination (5 animals per group). In combination arms, Compound of formula (I) was administered 2 hours prior to chemotherapy. Tumor growth was monitored twice weekly by caliper measurement and tumor volume calculated using the equation V=(L×W×W)/2, where V is tumor volume, L is tumor length (the longest tumor dimension) and W is tumor width (the longest tumor dimension perpendicular to L). During routine monitoring, the animals were checked for any effects of tumor growth and treatments on behavior such as mobility, food and water consumption, body weight gain/loss 3 times a week, eye/hair matting and any other abnormalities. Mortality and observed clinical signs were recorded for individual animals.

No significant body weight loss was observed throughout the study, indicating that the combination of Compound of formula (I) with carboplatin was well tolerated.

In this NCI-H446 model, treatment with either Compound of formula (I) or carboplatin in monotherapy led to no significant TGI effects compared to vehicle. However, the combination of Compound of formula (I) and carboplatin treatment led to a statistically significant TGI of 67% compared to vehicle (p=0.028). (FIG. 5).

### Example 4: In Vivo Efficacy and Tolerability in SC6 Patient Derived Xenoqraft (PDX) Model

Female athymic nude mice were inoculated subcutaneously with tumor fragments from SC6 PDX tumors. Animals were randomized when tumor volume reached approximately 60-200 mm³. Animals were treated with vehicle (0.5% methycellulose, 1% Tween 80 P.O. and saline I.V.), Compound of formula (I) formulated as a suspension in 0.5% methycellulose and 1% Tween 80 (30 mg/kg QD, P.O.), Carboplatin (25 mg/kg QW, I.P.) or etoposide (6 mg/kg QD [3 d on/4 d off], I.P.), administered over a 21 day cycle either alone or in combination (7 animals per group). In combination arms, Compound of formula (I) was administered 2 hours prior to chemotherapy. Tumor growth was monitored twice weekly by caliper measurement and tumor volume calculated using the equation V=(L×W×W)/2, where V is tumor volume, L is tumor length (the longest tumor dimension) and W is tumor width (the longest tumor dimension perpendicular to L). During routine monitoring, the animals were checked for any effects of tumor growth and treatments on behavior such as mobility, food and water consumption, body weight gain/loss 3 times a week, eye/hair matting and any other abnormalities. Mortality and observed clinical signs were recorded for individual animals.

In this PDX model, some transient body weight losses were observed immediately following Compound of formula (I) and etoposide combination treatment, however, all animals recovered quickly, remained on study and were evaluable, demonstrating that this combination was well tolerated.

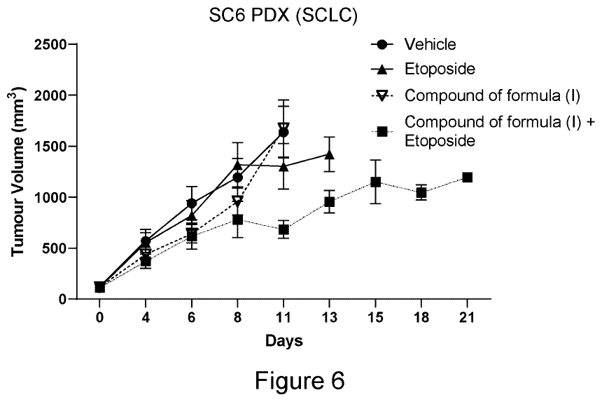

In this SC6 PDX model of SCLC, Compound of formula (I) in combination with etoposide induced an observable TGI compared to vehicle control of 34% at day 7 and 61% at day 11 (p=0.012). In the combination group, 4/7 mice remained below the maximum tumor volume ethical criteria at end of treatment (21 days) whereas all mice in all other groups had reached this criteria and were no longer on study (FIG. 6).

### Example 5a: Phase 1 Clinical Trial Involving Compound of Formula (I) (Referred to as the Study Drug)

The clinical trial is a multi-center, open-label, non-randomized, uncontrolled, dose-escalation study of Compound of formula (I) (the Study Drug) in combination with carboplatin and etoposide in adult participants with SCLC that recurred or progressed after previous standard platinum-based therapy, followed by an expansion in adult participants with SCLC that recurred or progressed after previous standard platinum-based therapy.

The clinical trial design is composed of 2 parts:
Part 1: dose escalation of oral, once-daily dosing of Study Drug given from Day 1 to Day 3 and from Day 8 to Day 10 in combination with etoposide (intravenous [IV] infusion) from Day 1 to Day 3 and with carboplatin (IV infusion) on Day 1 of a 21-day cycle. The study population for Part 1 consists of adult participants with histologically or cytologically confirmed small cell lung cancer (SCLC) that recurred or progressed after a minimum of 45 days since the last dose of prior standard platinum-based therapy. The Study Drug starting dose at dose level 1 (DL1) is at least one dose level lower than the highest dose of Study Drug found to be safe in the 101-study involving the Study Drug in combination with carboplatin.

The maximum tolerated dose (MTD) and/or recommended Phase 2 dose (RP2D) to be used in Part 2 is decided based on Safety Monitoring Committee (SMC) recommendation.
Part 2: the expansion starts after MTD and/or RP2D of Part 1 has been endorsed by the SMC. The study population for Part 2 consists of adult participants with histologically or cytologically confirmed SCLC that recurred or progressed after a minimum of 90 days since the last dose of prior standard platinum-based therapy. Participants are treated with Study Drug at the MTD and/or RP2D in combination with etoposide and carboplatin. Participant are followed for safety, anti-tumor activity and survival status until the end of the study.

In both Part 1 and Part 2, a treatment cycle is defined as 21 days.

The Primary Objectives and endpoints of the clinical trial include:

| Primary objective(s) | Endpoint(s) |
|---|---|
| Part 1 - Dose escalation | |
| To identify a recommended dose (RP2D | Incidence of DLTs, cumulative safety, and |
| and/or MTD) of Study Drug in combination | available data including but not limited to |
| with etoposide and carboplatin. | PK data, changes in laboratory values, vital |
| | signs, and ECGs. |
| Part 2 - Expansion | |
| To characterize the safety and tolerability of | Incidence of serious adverse events |
| Study Drug when administered in | (SAEs), incidence and severity of |
| combination with carboplatin and | treatment-emergent adverse events |
| etoposide. | (TEAEs) and laboratory abnormalities, |
| | graded according to National Cancer |
| | Institute Common Terminology Criteria for |
| | Adverse Events (NCI-CTCAE) criteria |
| | Incidence of treatment discontinuations |
| | and treatment modifications due to |
| | adverse events (AEs) and laboratory |
| | abnormalities |
| | Change in vital signs, electrocardiogram |
| | (ECG), echocardiogram, and Eastern |
| | Cooperative Oncology Group (ECOG) |
| | performance status (PS) |

The Secondary objectives and endpoints of the clinical trial include:

| Secondary objective(s) | Endpoint(s) |
|---|---|
| Part 1 - Dose escalation | |
| To characterize the safety and tolerability of | Incidence of SAEs, incidence and severity |
| Study Drug when administered in | of TEAEs and laboratory abnormalities, |
| combination with carboplatin and | graded according to NCI-CTCAE criteria |
| etoposide. | Incidence of treatment discontinuations |
| | and treatment modifications due to AEs |
| | and laboratory abnormalities |
| | Change in vital signs, ECG, |
| | echocardiogram and ECOG PS |
| To determine the pharmacokinetic (PK) | PK parameters of Study Drug: |
| profile of Study Drug in combination with | maximum plasma concentration (Cmax) |
| etoposide and carboplatin after C1D3 | time to Cmax (tmax) |
| administration and the last dose of the first | areas under the curve over 24h |
| cycle | (AUC24 h), up to the last measurable |
| | concentration (AUClast), up to infinity |
| | (AUCinf) |
| | apparent terminal half-life (t1/2) |
| | apparent total body clearance (CL/F) |
| | apparent volume of distribution (Vd/F) |
| | trough concentration at the end of Cycle 1 |
| | (Ctrough Cycle 1) |
| | others as deemed appropriate |
| To determine the PK profile of etoposide in | PK parameters of etoposide: Cmax, |
| combination with Study Drug and | AUC0-24 h, AUC0-46 h, AUCinf, CL, Vd and |
| carboplatin | t1/2 |
| To determine the PK profile of carboplatin | PK parameters of carboplatin: Cmax, |
| in combination with Study Drug and | AUCinf, CL, Vd and t1/2 |
| etoposide | |
| To investigate preliminary antitumor activity | Tumor response according to RECIST |
| of Study Drug when administered in | (Response Evaluation Criteria in Solid |
| combination with carboplatin and | Tumors) version 1.1 criteria: best overall |
| etoposide, in participants with SCLC that | response (BOR), overall response rate |
| recurred or progressed after a minimum of | (ORR), disease control rate (DCR), |
| 45 days since the last dose of prior | duration of response (DoR) |
| standard platinum-based therapy | Progression-free survival (PFS) |
| Part 2 - Expansion | |
| To investigate preliminary efficacy of Study | Tumor response according to RECIST |
| Drug when administered in combination | version 1.1 criteria: |
| with carboplatin and etoposide, in | ORR, DCR, DoR and BOR |
| participants with SCLC that recurred or | PFS |
| progressed after a minimum of 90 days | OS |
| since the last dose of prior standard | |
| platinum-based therapy. | |
| To confirm PK of Study Drug at the MTD | PK parameters of Study Drug (Cmax, |
| and/or RP2D in combination with etoposide | Ctrough, AUCs, cumulative AUC and other |
| and carboplatin | PK parameters as deemed appropriate) |

The study population of this clinical trial is as follows:
In Part 1 (Dose escalation): Adult participants (18 years old) with histologically or cytologically confirmed SCLC that recurred or progressed after a minimum of 45 days since the last dose of prior standard platinum-based therapy.
In Part 2 (Expansion): Adult participants (18 years old) with histologically or cytologically confirmed SCLC that recurred or progressed after a minimum of 90 days since the last dose of prior standard platinum-based therapy.

The inclusion criteria for this clinical trial include:
Histologically or cytologically confirmed SCLC
Non-bleeding tumor
Prior platinum-based chemotherapy (carboplatin and/or cisplatin)
   Part 1 (dose escalation): Recurrence or progression after a minimum of 45 days since the last dose of prior standard platinum-based therapy
   Part 2 (expansion): Recurrence or progression after a minimum of 90 days since the last dose of prior standard platinum-based therapy
Measurable disease per RECIST 1.1
Able and willing to undergo tumor biopsy unless an archived tumor sample is available
ECOG performance score 0-1
Life expectancy of at least 3 months in the best judgement of the Investigator
Adequate values for the following:
   bone marrow: absolute neutrophil count (ANC) ≥1500/μL, platelets ≥100,000/μL, hemoglobin ≥9 g/dL or 5.6 mmol/L, and no blood transfusions within the last 2 weeks of study treatment initiation
   liver biochemistry: AST and ALT ≤2.5×upper limit of normal (ULN) or ≤5×ULN for participants with liver metastases; serum total bilirubin ≤1.5 ULN and direct/indirect bilirubin ≤ULN; alkaline phosphatase (ALP)<2.5×ULN or ≤5×ULN for participants with liver metastases
   renal function: calculated creatinine clearance ≥50 mL/minute (min) (as determined by the Chronic Kidney Disease—Epidemiology Collaboration [CKD EPI] formula)
   coagulation status: international normalized ratio (INR) or prothrombin time ≤1.5×ULN; activated partial thromboplastin time (aPTT) ≤1.5×ULN.

The exclusion criteria for this clinical trial include:
Prior treatment with more than 1 line of therapy for extensive SCLC disease, unless participant has received 1 line of prior immunotherapy without chemotherapy. In this case, 2 lines of therapy would be allowed (one of the lines would need to be platinum-based chemotherapy).
Current use of an investigational agent or medical device.
History of active second malignancies requiring therapy in the last 6 months, with the exception of superficial bladder cancers, ductal carcinoma in situ or other carcinomas in situ, and non-melanoma skin cancers (basal cell/squamous cell skin cancer) that have been treated surgically.
Clinically significant gastrointestinal abnormality that could affect the absorption of orally administered drugs (e.g., ulcerative diseases, gastrointestinal dysfunction, uncontrolled nausea, vomiting, diarrhea, malabsorption syndrome, major resection of the small bowel or total gastrectomy, or inflammatory bowel disease).
Radiographic findings of interstitial lung disease (ILD) that are considered clinically significant (e.g., symptomatic)
Known infection requiring the systemic use of an antibiotic or antiviral agent.
Immunization with live or live-attenuated vaccine within 28 days prior to first dose of study treatment.
Pregnancy or breast-feeding.
Inability or unwillingness to swallow oral medications.
Unresolved AEs or toxicities due to previous treatments >Grade 1. Note: participants with sGrade 2 alopecia or endocrinopathies controlled by replacement therapy (e.g., hypothyroidism due to immune checkpoint inhibitors) are exceptions and may qualify for the study.
Hypersensitivity to Study Drug, etoposide or carboplatin, or any of the excipients found in the formulation for Study Drug, etoposide, or carboplatin.

The investigational products used in this clinical trial are as follows:
Study Drug is formulated as 10 mg mini-tablets included in hard gelatin capsules. Study Drug is taken orally on an empty stomach (i.e., participants must be fasting [only water permitted] for 4 hours [h] before and 2 h after intake). For Part 1, the starting dose at DL1 is at least one dose level lower than the highest dose of Study Drug found to be safe in the 101-study involving the Study Drug in combination with carboplatin.
Etoposide is a concentrate for solution for IV infusion. Participants receive etoposide for 3 consecutive days during each cycle, i.e., on Day 1, Day 2 and Day 3. Etoposide infusion starts 5-15 min after the morning dose of Study Drug at a dose of 100 mg/m2, and infusion duration is 30 min to 1 h.
Carboplatin is a concentrate for solution for IV infusion. Participants receive carboplatin on Day 1 of each cycle. Carboplatin infusion starts within 30 min after the end of the etoposide infusion at a dose based on the AUC 5 mg/mL·min. The infusion duration is 30 min to 1 h.

Treatment duration for each participant is until either progression of disease, unacceptable toxicity, participant withdrawal, discontinuation as per Investigator's decision, or end of the study. The overall end of study occurs at the time of data cut-off for the main analysis, namely when the last enrolled participant has received 12 months of treatment or the last on-study participant has discontinued, whichever happens first.

It is estimated that approximately 54 evaluable patients are enrolled in the clinical trial.

### Example 5b: Phase 1 Clinical Trial Involving Compound of Formula (I) (Referred to Herein as the Study Drug)

A clinical trial will be conducted, which is a Phase 1, open-label, multicenter study composed of 2 parts, a dose escalation and an expansion, to assess safety and preliminary antitumor activity of Study Drug in combination with carboplatin and etoposide in adult participants with small cell lung cancer that recurred or progressed after previous standard platinum-based therapy.

### Part 1 (Dose Escalation):

Dose escalation of oral, once-daily dosing of Study Drug from Day 1 to Day 3 and from Day 8 to Day 10 of a 21-day cycle, in combination with etoposide (intravenous [IV] infusion) from Day 1 to Day 3 and with carboplatin (IV infusion) on Day 1. The study population for Part 1 will consist of adult participants with histologically or cytologically confirmed small-cell lung cancer (SCLC) that recurred or progressed after a minimum of 45 days since the last dose of prior standard platinum-based therapy. The Study Drug starting dose at dose level 1 (DL1) will be 200 mg based on the available data from the 101-study involving the Study Drug in combination with carboplatin. Participants will be enrolled into cohorts consisting of at least 3 participants treated at the same dose level.

During Part 1, dose recommendations on Study Drug dose escalation in combination with etoposide and carboplatin will be made by the Safety Monitoring Committee (SMC). The maximum tolerated dose (MTD) and/or recommended Phase 2 dose (RP2D) to be used in Part 2 is decided based on SMC recommendation.

### Part 2 (Expansion):

The expansion will start after RP2D of Part 1 has been endorsed by the SMC. The study population for Part 2 will consist of adult participants with histologically or cytologically confirmed SCLC that recurred or progressed after a minimum of 90 days since the last dose of prior standard platinum-based therapy. Participants will be treated with Study Drug at the RP2D in combination with etoposide and carboplatin.

In part 2, participants will be followed for safety until the safety follow-up visit occurs (and afterwards for all serious adverse events [SAEs] and related AEs until the End of Study (EOS)), and for antitumor activity until disease progression. Follow-up for survival status will be performed until withdrawal of consent, death, or the end of the study, whichever occurs first. In addition, quality of life will be assessed over the course of the study until the EOT visit.

In both Part 1 and Part 2, a treatment cycle is defined as 21 days.

### Abbreviations Used in the Tables and Text of this Example 5b

AE=adverse event;
AUC=area under the concentration vs time curve;
AUC₂₄=area under the curve up to 24 h;
AUC_{inf}=area under the curve up to infinity;
AUCₗₐₛₜ=area under the curve up to the last measurable concentration;BOR=best overall response;
CL/F=apparent total body clearance;
Cₘₐₓ=maximum plasma concentration; CR=complete response;
CSF=cerebrospinal fluid;
C_{trough}=trough plasma concentration; CV=coefficient of variation;
DCR=disease control rate;
DMET=Drug Metabolizing Enzymes and Transporters;
DOR=duration of response;
DLT=dose-limiting toxicity;
ECG=electrocardiogram;
ECOG=Eastern Cooperative Oncology Group;
EOT=End of Treatment;
FACT-G=Functional Assessment of Cancer Therapy—General;
FACT-L=Functional Assessment of Cancer Therapy—Lung;
NCI-CTCAE 5.0=National Cancer Institute Common Terminology Criteria for
Adverse Events, version 5.0;
OR=objective response;
ORR=objective response rate;
OS=overall survival;
PFS=progression-free survival;
PK=pharmacokinetic(s);
PR=partial response;
PS=performance status;
QoL=quality of life
RECIST 1.1=Response Evaluation Criteria in Solid Tumours, version 1.1;
RP2D=recommended Phase 2 dose;
SCLC=small-cell lung cancer;
SD=standard deviation;
TEAE=treatment-emergent adverse event;
T_{1/2}=apparent terminal half-life
tₘₐₓ=time to Cₘₐₓ;
TOI=Trial Outcome Index;
Vd=volume of distribution;
Vd/F=apparent Vd

The Primary Objectives and Estimands (including endpoints) of the clinical trial include:

| Objectives | Estimands | Ref. # |
|---|---|---|
| Primary Objectives | | |
| Part 1 - Dose escalation | | |
| To identify the recommended | Endpoint: Incidence of DLTs | 1 |
| dose (RP2D) and to | Population: Adult participants with histologically or | |
| characterize the safety and | cytologically confirmed SCLC that recurred or | |
| tolerability of Study Drug in | progressed after prior standard platinum-based | |
| combination with carboplatin | therapy | |
| and etoposide | Treatment: Study Drug in combination with carboplatin | |
| | and etoposide as further defined in this Example 5b | |
| | Population-level summary measure: DLT rate as a | |
| | function of dose | |
| | Note: To identify the RP2D, other available data from | |
| | secondary and exploratory endpoints (e.g., PK data, | |
| | antitumor activity data) may be taken into consideration. | |
| | Endpoint: | 2 |
| | Incidence and severity of TEAEs, serious TEAEs, | |
| | TEAEs leading to death, and TEAEs leading to | |
| | treatment discontinuation and/or dose | |
| | modifications, graded according to NCI-CTCAE | |
| | 5.0 | |
| | Laboratory parameters and related severity based | |
| | on NCI-CTCAE 5.0 | |
| | Incidence of treatment discontinuations and | |
| | treatment modifications due to TEAEs and | |
| | laboratory abnormalities | |
| | Changes from baseline in parameters of vital | |
| | signs, ECG, echocardiogram, and ECOG PS | |
| | Population: See Ref. #1 | |
| | Treatment: Study Drug in combination with carboplatin | |
| | and etoposide | |
| | Population-level summary measures: | |
| | For incidence of TEAEs: Number of events, number | |
| | of participants with events, percentages of | |
| | participants with at least one event. For severity: | |
| | Distribution of severity. | |
| | For changes in laboratory values from baseline: | |
| | Proportion of participants with laboratory | |
| | abnormalities and distribution of grading for | |
| | laboratory abnormalities. | |
| | Proportion of treatment discontinuations and | |
| | treatment modifications | |
| | Changes from baseline in parameters of vital signs, | |
| | ECG, echocardiogram, and ECOG PS. | |
| | Note: To identify the RP2D, other available data from | |
| | secondary and exploratory endpoints (e.g., PK data, | |
| | antitumor activity data) may be taken into consideration. | |
| Part 2 - Expansion | | |
| To characterize the safety | Endpoint: | 3 |
| and tolerability of Study Drug | Incidence and severity of TEAEs, serious | |
| at the recommended dose | TEAEs, TEAEs leading to death, and TEAEs | |
| when administered in | leading to treatment discontinuation and/or | |
| combination with carboplatin | dose modifications, graded according to NCI- | |
| and etoposide | CTCAE 5.0 | |
| | Laboratory parameters and related severity | |
| | based on NCI-CTCAE 5.0 | |
| | Incidence of treatment discontinuations and | |
| | dose modifications due to TEAEs and | |
| | laboratory abnormalities | |
| | Changes from baseline in parameters of vital | |
| | signs, ECG, echocardiogram, and ECOG PS | |
| | Population: Adult participants with histologically or | |
| | cytologically confirmed SCLC that recurred or | |
| | progressed after prior standard platinum-based | |
| | therapy | |
| | Treatment: Study Drug in combination with | |
| | carboplatin and etoposide | |
| | Population-level summary measures: | |
| | For incidence of TEAEs: Number of events, | |
| | number of participants with events, | |
| | percentages of participants with at least one | |
| | event. For severity: Distribution of severity. | |
| | For changes in laboratory values from | |
| | baseline: Proportion of participants with | |
| | laboratory abnormalities and distribution of | |
| | grading for laboratory abnormalities. | |
| | Proportion of treatment discontinuations and | |
| | treatment modifications | |
| | Changes from baseline in parameters of vital | |
| | signs, ECG, echocardiogram, and ECOG PS. | |

The Secondary objectives and Estimands (including endpoints) of the clinical trial include:

| Secondary Objectives | Estimands | Ref. # |
|---|---|---|
| Part 1 - Dose escalation | | |
| To determine the PK of Study | Endpoint: PK profile and parameters of Study Drug | 4 |
| Drug (and of its metabolite | and its metabolite on Day 3 and Day 10 of the first | |
| N³²-desmethyl-Study Drug) in | cycle, including but not limited to: | |
| combination with etoposide | Cₘₐₓ | |
| and carboplatin | tₘₐₓ | |
| | AUC_{24 h}, AUCₗₐₛₜ, AUC_{inf} | |
| | t_{1/2} | |
| | CL/F | |
| | V_{d}/F | |
| | C_{trough C1} | |
| | Population: See Ref. #1 | |
| | Treatment: Study Drug in combination with carboplatin | |
| | and etoposide | |
| | In case of no administration of Study drug or event | |
| | such as vomiting or co-administration of a prohibited | |
| | medication, the patient might not be considered for PK | |
| | assessment. | |
| | Population-level summary measures: Mean, | |
| | geometric mean, median, SD, CV, min, max, and | |
| | geometric CV of plasma concentrations and PK | |
| | parameters | |
| To determine the PK of | Endpoint: PK profile and parameters of etoposide | 5 |
| etoposide in combination with | including but not limited to: Cₘₐₓ, AUC_{24 h}, AUCₗₐₛₜ, | |
| Study Drug and carboplatin | AUC_{inf}, CL, V_{d}, and t_{1/2} | |
| | Population/In case of/Treatment/Population-level | |
| | summary measures: See Ref. #4 | |
| To determine the PK of | Endpoint: PK profile and parameters of carboplatin | 6 |
| carboplatin in combination | including but not limited to: Cₘₐₓ, AUC_{24 h}, AUCₗₐₛₜ, | |
| with Study Drug and | AUC_{inf}, CL, V_{d}, and t_{1/2} | |
| etoposide | Population/In case of/Treatment/Population-level | |
| | summary measures: See Ref. #4 | |
| To investigate preliminary | Endpoint: Tumor response according to RECIST 1.1 | 7 |
| antitumor activity of Study | criteria: BOR, OR, disease control, DOR, and PFS | |
| Drug when administered in | Population: Participants with SCLC that recurred or | |
| combination with carboplatin | progressed after a minimum of 45 days since the last | |
| and etoposide | dose of prior standard platinum-based therapy | |
| | Treatment: Study Drug in combination with carboplatin | |
| | and etoposide. | |
| | Population-level summary measures: | |
| | Estimates of the ORR and DCR | |
| | Distribution of BOR | |
| | Kaplan-Meier estimates for time-to-event endpoints | |
| | (DOR, PFS) | |
| Part 2- Expansion | | |
| To assess the ORR of Study | Endpoint: OR, defined as tumor response (CR or PR) | 8 |
| Drug at the recommended | according to RECIST 1.1 criteria | |
| dose when administered in | Population: Participants with SCLC that recurred or | |
| combination with carboplatin | progressed after a minimum of 90 days since the last | |
| and etoposide | dose of prior standard platinum-based therapy | |
| | Treatment: Study Drug in combination with carboplatin | |
| | and etoposide | |
| | Population-level summary measure: | |
| | Estimate of the ORR | |
| To investigate additional | Endpoint: Tumor response according to RECIST 1.1 | 9 |
| parameters of preliminary | criteria: BOR, DC, DOR, PFS, and OS | |
| antitumor activity of Study | Population: See Ref. #8 | |
| Drug at the recommended | Treatment: Study Drug in combination with carboplatin | |
| dose when administered in | and etoposide | |
| combination with carboplatin | Population-level summary measures: | |
| and etoposide | Estimate of DCR | |
| | Distribution of BOR | |
| | Kaplan-Meier estimates for time-to-event endpoints | |
| | (DOR, PFS, OS) | |
| To confirm the PK of Study | Endpoint: PK parameters of Study Drug and its | 10 |
| Drug (and its metabolite) at | metabolite (including Cₘₐₓ, C_{trough}, AUC_{24 h}) | |
| the recommended dose in | Population/Treatment/Population-level summary | |
| combination with etoposide | measure/In case of: See Ref. #4 | |
| and carboplatin | | |

The exploratory objectives of the clinical trial include:

| Exploratory Objectives | Estimands | Ref. # |
|---|---|---|
| Part 1 - Dose escalation | | |
| and Part 2 - Expansion | | |
| To explore brain penetration | Endpoint: | 11 |
| of Study Drug (and its | Concentrations of Study Drug (and its metabolite) | |
| metabolite) | in CSF and plasma samples collected at the same | |
| | time point during treatment (i.e., on Cycle 1 Day 10 | |
| | and/or whenever a CSF sample is collected) | |
| | Ratio of unbound Study Drug concentrations in | |
| | CSF and plasma | |
| | Population-level summary measure: Descriptive | |
| | analysis between CSF and plasma concentrations | |
| | and ratios | |
| To explore relationships | Endpoint: Safety and/or antitumor activity parameters | 14 |
| between Study Drug (and its | associated with PK parameters of Study Drug and its | |
| metabolite) plasma exposure | metabolite | |
| and safety and antitumor | Population-level summary measures: Correlation | |
| activity | measure and/or descriptive analysis between safety | |
| | and/or antitumor activity parameters and PK | |
| | parameters | |
| Part 2 - Expansion | | |
| To assess QoL of participants | Endpoint: | 15 |
| receiving Study Drug in | Change from baseline in FACT-G score, Lung Cancer | |
| combination with carboplatin | Subscale score, TOI, and FACT-L Total Score | |
| and etoposide | (analyzed separately) | |
| | Time to deterioration (time to first deterioration and | |
| | time to confirmed deterioration) in FACT-G score, | |
| | Lung Cancer Subscale score, TOI, and FACT-L Total | |
| | Score (analyzed separately) | |

The study population of this clinical trial is as follows:
Part 1 (dose escalation): Adult participants (18 years old) with histologically or cytologically confirmed SCLC that recurred or progressed after a minimum of 45 days since the last dose of prior standard platinum-based therapy.
Part 2 (expansion): Adult participants (18 years old) with histologically or cytologically confirmed SCLC that recurred or progressed after a minimum of 90 days since the last dose of prior standard platinum-based therapy.

The inclusion criteria of this clinical trial include:
Histologically or cytologically confirmed SCLC.
Tumor that is not bleeding.
Prior platinum-based chemotherapy (carboplatin and/or cisplatin)
   Part 1 (dose escalation): Recurrence or progression after a minimum of 45 days since the last dose of prior standard platinum-based therapy
   Part 2 (expansion): Recurrence or progression after a minimum of 90 days since the last dose of prior standard platinum-based therapy.
Only 1 prior systemic treatment (i.e., platinum-based chemotherapy with or without immunotherapy) for extensive-stage (ES) SCLC is allowed. Exception: Participants who received prior lurbinectedin as monotherapy in addition to 1 prior platinum-based chemotherapy treatment are also considered eligible.
Measurable disease per RECIST 1.1.
Willingness and ability to undergo tumor biopsy unless an archived tumor sample is available.
ECOG performance status of 0-1.
Life expectancy of at least 3 months in the best judgment of the Investigator.
Adequate values for the following:
   Bone marrow: Absolute neutrophil count (ANC) ≥1500/μL, platelets ≥100 000/μL, hemoglobin 9 g/dL or 5.6 mmol/L, and no blood transfusions within the last 2 weeks before study treatment initiation.
   Liver biochemistry: Aspartate aminotransferase (AST) and alanine aminotransferase (ALT)≤2.5×upper limit of normal (ULN), or ≤5×ULN for participants with liver metastases; serum total bilirubin ≤1.5×ULN; alkaline phosphatase (ALP) <2.5×ULN or ≤5×ULN for participants with liver metastases.
Renal function: Calculated creatinine clearance ≥50 mL/minute (min) (as determined by the Chronic Kidney Disease—Epidemiology Collaboration [CKD EPI] formula).
Coagulation status: International Normalized Ratio (INR) or prothrombin time ≤1.5×ULN; activated partial thromboplastin time (aPTT) ≤1.5×ULN.
Willing to practice highly effective methods of contraception.

The exclusion criteria of this clinical trial include:
Use of an investigational agent or medical device within 28 days prior to first dose of study treatment.
History of other malignancies requiring active treatment in the last 2 years prior to the first dose of study treatment, except for superficial bladder cancers, ductal carcinoma in situ or other carcinomas in situ, and non-melanoma skin cancers (basal cell/squamous cell skin cancer) that have been treated with curative intent.
Brain metastases unless they are asymptomatic and have been stable for at least 1 month (with a maximum steroid dose of 10 mg/day of prednisone or 4 mg/day of dexamethasone) and have not required active treatment in the last month prior to first dose of study treatment.
History of myocardial infarction or stroke within 6 months prior to first dose of study treatment, congestive heart failure greater than New York Heart Association (NYHA) class II, unstable angina pectoris, unexplained recurrent syncope, cardiac arrhythmia requiring treatment, known family history of sudden death from cardiac-related causes before the age of 50, or any cardiotoxicity experienced after previous chemotherapy.
Left ventricular ejection fraction (LVEF) below 55%.
Heart rate-corrected QT interval (QTc) using Fridericia's formula (QTcF) >450 ms, history of congenital long QT syndrome, or clinically significant conduction abnormality, or any conduction abnormality that may increase the risk of torsades de pointes (TdP).
Concomitant use of a drug with a known risk of TdP/QTc prolongation or of any drug(s) described in the prohibited medications section of the protocol. If such a drug has been used by the participant, a wash-out period of at least 5 half-lives of the drug must occur before first administration of study treatment.
Concomitant use of a drug or herbal product that is an inhibitor or inducer of cytochrome P450 (CYP) 3A4, a strong inhibitor of CYP2D6, or of any drug(s) (such as proton pump inhibitors, H2 receptor antagonists, etc.) described in the prohibited medications section of the protocol. If such a drug has been used by the participant, a wash-out period of at least 5 half-lives of the drug must occur before first administration of study treatment.
Clinically significant gastrointestinal abnormality that could affect the absorption of orally administered drugs (e.g., ulcerative diseases, gastrointestinal dysfunction, uncontrolled nausea, vomiting, diarrhea, malabsorption syndrome, major resection of the small bowel or total gastrectomy, or inflammatory bowel disease).
Major surgery 54 weeks prior to first dose of study treatment or incomplete recovery from the surgical procedure at the time of the first dose of study treatment.
Exposure to high levels of ultraviolet (UV) light, for example occupational exposure to sunlight or sunbathing.
Radiographic findings showing tumor involvement with large blood vessels or poor demarcation from them with increased risk for bleeding.
Radiographic findings of interstitial lung disease (ILD) that are considered clinically significant (e.g., symptomatic).
Uncontrolled pleural effusion, pericardial effusion, or ascites requiring repeated drainage.
Any infection requiring the systemic use of an antibiotic or antiviral agent.
Known hepatitis C virus (HCV), hepatitis B virus (HBV), or human immunodeficiency virus (HIV) infection. Participants with past infections that have been cured may be enrolled.
Immunization with live or live-attenuated vaccine within 28 days prior to first dose of study treatment.
Inability or unwillingness to swallow oral medications.
Chemotherapy, monoclonal antibodies/biologics, or radiotherapy with curative intent within 28 days prior to first dose of study treatment. Palliative radiation (e.g., for pain relief) is allowed up to 1 week prior to study treatment start.
Unresolved AEs or toxicities due to previous treatments >Grade 1. Note: Participants with ≤Grade 2 alopecia or endocrinopathies controlled by replacement therapy (e.g., hypothyroidism due to immune checkpoint inhibitors) are exceptions and may qualify for the study.
Hypersensitivity to Study Drug, etoposide or carboplatin, or any of the excipients found in the formulations for Study Drug, etoposide, or carboplatin. If a prior hypersensitivity to carboplatin has been observed, but a successful desensitization was performed for the participant, he or she may be eligible for the study.
Prior exposure to any WEE1 inhibitor.

The investigational products, doses and modes of administration used in this clinical trial are as follows:

The Study Drug is formulated as 20, 30, 60, 100, 130, and 150 mg hard gelatin capsules. The capsules contain different quantities of 10 mg uncoated mini-tablets (excipients are as follows: Microcrystalline cellulose, anhydrous lactose, hydroxypropylcellulose, sodium starch glycolate, anhydrous colloidal silica, and magnesium stearate). Intermediate capsule strengths may be produced to cover alternative dose levels.

Participants will receive Study Drug for 3 consecutive days per week during the first 2 weeks of each 21-day cycle (on Day 1, Day 2, and Day 3 and Day 8, Day 9, and Day 10). Study drug is taken orally on an empty stomach (i.e., participants must be fasting [only water permitted] for at least 4 hours before and 2 hours after intake). In Part 1, the starting dose (DL1) will be 200 mg per day.

Etoposide is a concentrate for solution for IV infusion. Participants will receive etoposide for 3 consecutive days during each cycle, i.e., on Day 1, Day 2, and Day 3. Etoposide infusion will start 5-15 min after the morning dose of Study Drug at a dose of 100 mg/m2, and infusion duration will be 30 min to 1 h.

Carboplatin is a concentrate for solution for IV infusion. Participants will receive carboplatin on Day 1 of each cycle. Carboplatin infusion will start within 30 min after the end of the etoposide infusion at a dose based on the AUC 5 mg/mL·min. The infusion duration will be 30 min to 1 h.

The treatment duration for each participant will be until progression of the disease, unacceptable toxicity, participant withdrawal, discontinuation as per Investigator's decision, death, or the overall end of the study.

It is estimated that up to approximately 54 evaluable participants will be enrolled in the study.

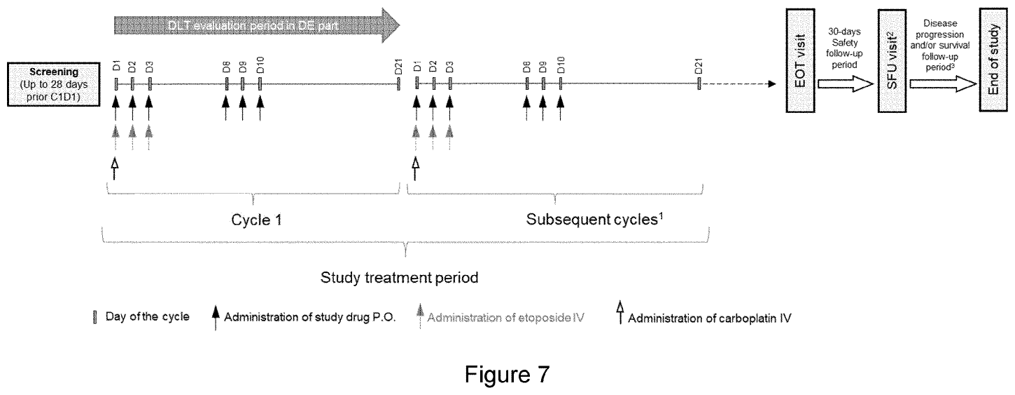

FIG. 7 provides a study diagram illustrating the schedule of administration of triple combination in both the dose escalation (DE) and the expansion parts of the clinical trial. In FIG. 7, the following abbreviations are used: AE=adverse event; C=Cycle; D=Day; DE=Dose escalation; DLT=dose-limiting toxicity; EOS=End of study; EOT=End of treatment; P.O.=per os; SAE=Serious adverse event; SFU=Safety follow-up;

In FIG. 7, the footnotes specify the following:
¹until progressive disease/toxicity
²after the end of SFU period and until EOS for the participant, all SAEs (regardless of causality assessment) and AEs considered to be related to study treatment as per investigator assessment will be collected
³Survival follow-up only for the expansion part

A cycle is defined as 21 days.

### Example 6: Activity of Compound of Formula (I) as Monotherapy, and in Combination with Carboplatin and Etoposide in SCLC Orqanoid Models

SCLC organoids from 16 models were prepared as follows: SCLC organoid models were expanded in 6-well plates, each well containing 2 ml of organoid medium. On the day of organoid seeding 20 μL 100× Dispase solution was added to each well and the plates were incubated at 37° C. for 30 min. Organoids were passed through a pre-wet 100 μm filter into a 50 ml plastic tube and subsequently further filtered through a wet 20 μm filter. Organoids were resuspended in culture media and counted. Matrigel was added to a final concentration of 5% v/v and 40 μL of cell suspensions add to each well of a 384-well plate by Multidrop dispenser.

The treatments tested for each organoid model were as follows:
(a) Compound of Formula (I) in monotherapy,
(b) carboplatin and etoposide (in a 5:1 ratio, considered as a single treatment), or
(c) Compound of Formula (I) in combination with carboplatin and etoposide, using the following concentrations in a matrix format: compound of Formula (I) in 6 concentrations from 0.047 to 1.5 μM, carboplatin in 6 concentrations from 0.156 to 5 μM, and etoposide in 6 concentrations from 0.031 to 1 μM)). Thus, for the carboplatin and etoposide treatment (considered as a single treatment in this experiment), the lowest concentration of carboplatin was combined with the lowest concentration of etoposide in a 5:1 ratio, the 2^{nd} lowest doses of both reagents were combined, etc., while maintaining the 5:1 ratio. Then, each of these combinations were used either alone (carboplatin/etoposide treatment) or in further combination with each concentration of Compound of Formula (I).

The screening plates were incubated with treatments for 5 days. All treatments were performed in technical triplicates and biological replicates.

Plates were assayed for luminescent Cell Titer Glow (CTG) signal by adding 40 μL of CTG 3D per well by Multidrop dispenser, mixing for 5 mins and incubating for 30 min at room temperature in the dark. Luminescence signal was then measured on Envision plate reader.

The surviving and inhibition rates were calculated as follows for each treatment:

$$surviving rate ( \% ) = ( {Lum}_{Test article} - {Lum}_{Medium control} ) / ( {Lum}_{Vehicle control} - {Lum}_{Medium control} ) \times 100 \% .$$

$$inhibition rate ( \% ) = 100 - surviving rate ( \% ) .$$

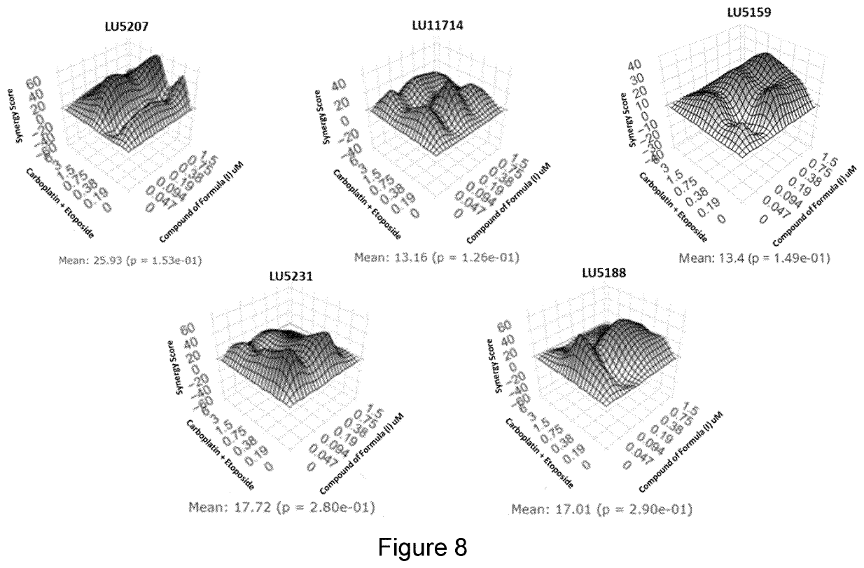

For synergy calculations and creation of heatmaps, the Bliss independence model was used on synergyfinder.org (Zheng, S.; Wang, W.; Aldahdooh, J.; Malyutina, A.; Shadbahr, T.; Tanoli Z; Pessia, A.; Jing, T. SynergyFinder Plus: Toward Better Interpretation and Annotation of Drug Combination Screening Datasets. Genomics, Proteomics & Bioinformatics 2022, in press. doi:10.1016/j.gpb.2022.01.004) considering carboplatin/etoposide as a single treatment vs Compound of Formula (I). 9 out of 16 models had bliss synergy scores >5 between Compound of Formula (I) and the carboplatin/etoposide treatment. Surface plots of bliss independence model in synergism analysis are presented in FIG. 8 for 5 of these models with a highly significant synergy score >10.

In addition, 12 out of 16 SCLC organoids were sensitive to treatment with Compound of Formula (I) alone (i.e. as monotherapy), as demonstrated by IC50 values lower than 1.5 uM (corresponding to plasma concentrations achieved in humans), and a maximum inhibition rate (cell death rate) greater than 60% at 1.5 uM (the highest dose tested for Compound of Formula (I)).

1. A method for treating small cell lung cancer (SCLC) in a patient in need thereof comprising administering to the patient a WEE1 inhibitor.

2. The method according to claim 1, wherein the WEE1 inhibitor is a compound of formula (I) 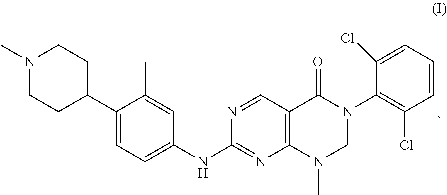 [SMILES: Cc1cc(Nc2ncc3c(n2)N(C)CN(c2c(Cl)cccc2Cl)C3=O)ccc1C1CCN(C)CC1]
or a pharmaceutically acceptable salt thereof.

3. The method according to claim 2, wherein said WEE1 inhibitor is used in combination with carboplatin and/or etoposide.

4. The method according to claim 3, wherein said WEE1 inhibitor is used in combination with carboplatin and etoposide.

5. The method according to claim 3, wherein the SCLC has recurred or progressed after initial or prior SCLC treatment.

6. The method according to claim 5, wherein the SCLC has recurred or progressed
a) 45 days or more since a last dose of standard platinum-based therapy, or
b) 90 days or more since a last dose of standard platinum-based therapy.

7. The method according to claim 3, wherein the patient is naïve of any prior SCLC treatment.

8. The method according to claim 3, wherein the WEE1 inhibitor is administered orally.

9. The method according to claim 3, wherein the WEE1 inhibitor is administered
a) on days 1, 2 and 3 of a 21-day cycle, or
b) on days 1, 2, 3 and 8, 9, 10 of a 21-day cycle.

10. The method according to claim 3, wherein the WEE1 inhibitor is administered
a) at a dose ranging from 150 to 720 mg per treatment day or ranging from 100 to 520 mg per treatment day, and/or
b) as a single dose on a treatment day.

11. The method according to claim 3, wherein
a) the WEE1 inhibitor is administered at approximately the same time on each treatment day, and/or
b) the WEE1 inhibitor is administered after fasting, and/or
c) WEE1 inhibitor administration is followed by fasting.

12. The method according to claim 3, wherein etoposide is administered by infusion.

13. The method according to claim 12, wherein etoposide is administered
a) on days 1, 2 and 3 of a 21-day cycle, and/or
b) at a dose ranging from 70 to 100 mg/m2, per infusion.

14. The method according to claim 3, wherein etoposide is administered orally.

15. The method according to claim 14, wherein etoposide is administered
a) on days 1 to 5 of a 21-day cycle, at a dose ranging from 100 to 200 mg/m2/day, or
b) at a dose ranging from 100 to 200 mg/m2/day on days 1 to 5 every 3 to 4 weeks, or
c) at a dose of 200 mg/m2/day on days 1, 3 and 5 every 3 to 4 weeks.

16. The method according to claim 3, wherein carboplatin is administered
a) by infusion at a dose corresponding to an AUC ranging from 2 to 6 mg/ml×min, according to the Calvert formula, and/or
b) on day 1 of a 21-day cycle.

17. The method according to claim 3, wherein on a WEE1 inhibitor treatment day, administration of the WEE1 inhibitor precedes administration of etoposide and/or carboplatin.

18. The method according to claim 3, wherein on a WEE1 inhibitor treatment day,
a) etoposide administration is initiated about 5 minutes to 1 hour after WEE1 inhibitor administration, and/or
b) carboplatin administration is initiated about 5 minutes to 1 hour after WEE1 inhibitor administration, or after the end of etoposide administration.

19. The method according to claim 9, wherein the WEE1 inhibitor is administered over 1, 2, 3, 4, 5, 6 or more cycles.

20. A pharmaceutical composition comprising the compound of formula (I), or a pharmaceutically acceptable salt thereof,

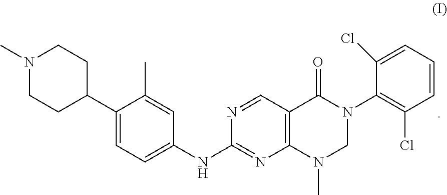 [SMILES: Cc1cc(Nc2ncc3c(n2)N(C)CN(c2c(Cl)cccc2Cl)C3=O)ccc1C1CCN(C)CC1]

21. A kit comprising the compound of formula (I), or a pharmaceutically acceptable salt thereof, 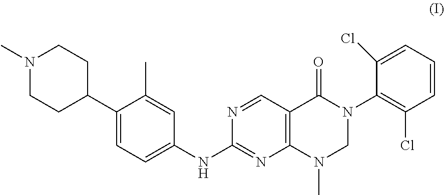 [SMILES: Cc1cc(Nc2ncc3c(n2)N(C)CN(c2c(Cl)cccc2Cl)C3=O)ccc1C1CCN(C)CC1]
and instructions for use, wherein said WEE1 inhibitor is used in combination with carboplatin and/or etoposide in treating small cell lung cancer (SCLC) in a patient in need thereof.

22. (canceled)

23. (canceled)

24. The method according to claim 4, wherein the SCLC has recurred or progressed after initial or prior SCLC treatment.

25. The method according to claim 4, wherein the patient is naïve of any prior SCLC treatment.
